SEQUENCE LISTING (5 sequences)

SEQ ID NO: 1 - protein, 1218 aa, Homo sapiens [sequence omitted]

SEQ ID NO: 2 - protein, 1218 aa, Mus musculus
MRSPRTRGRPGRPLSLLLALLCALRAKVCGASGQFELEILSMQNVNGELQNGNCCGGVRNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPDCNKAICRQGCSPKHGSCKLPGDCRCQYGWQGLYCDKCIPHPGCVHGTCNEPWQCLCETNWGGQLCDKDLNYCGTHQPCLNRGTCSNTGPDKYQCSCPEGYSGPNCEIAEHACLSDPCHNRGSCKETSSGFECECSPGWTGPTCSTNIDDCSPNNCSHGGTCQDLVNGFKCVCPPQWTGKTCQLDANECEAKPCVNARSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTTCEVIDSCTVAMASNDTPEGVRYISSNVCGPHGKCKSQSGGKFTCDCNKGFTGTYCHENINDCESNPCKNGGTCIDGVNSYKCICSDGWEGAHCENNINDCSQNPCHYGGTCRDLVNDFYCDCKNGWKGKTCHSRDSQCDEATCNNGGTCYDEVDTFKCMCPGGWEGTTCNIARNSSCLPNPCHNGGTCVVNGDSFTCVCKEGWEGPICTQNTNDCSPHPCYNSGTCVDGDNWYRCECAPGFAGPDCRININECQSSPCAFGATCVDEINGYQCICPPGHSGAKCHEVSGRSCITMGRVILDGAKWDDDCNTCQCLNGRVACSKVWCGPRPCRLHKSHNECPSGQSCIPVLDDQCFVRPCTGVGECRSSSLQPVKTKCTSDSYYQDNCANITFTFNKEMMSPGLTTEHICSELRNLNILKNVSAEYSIYIACEPSLSANNEIHVAISAEDIRDDGNPVKEITDKIIDLVSKRDGNSSLIAAVAEVRVQRRPLKNRTDFLVPLLSSVLTVAWVCCLVTAFYWCVRKRRKPSSHTHSAPEDNTTNNVREQLNQIKNPIEKHGANTVPIKDYENKNSKMSKIRTHNSEVEEDDMDKHQQKVRFAKQPVYTLVDREEKAPSGTPTKHPNWTNKQDNRDLESAQSLNRMEYIV

SEQ ID NO: 3 - protein, 1219 aa, Rattus norvegicus
MRSPRTRGRPGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPECNKAICRQGCSPKHGSCKLPGDCRCQYGWQGLYCDKCIPHPGCVHGTCNEPWQCLCETNWGGQLCDKDLNYCGTHQPCLNRGTCSNTGPDKYQCSCPEGYSGPNCEIAEHACLSDPCHNRGSCKETSSGFECECSPGWTGPTCSTNIDDCSPNNCSHGGTCQDLVNGFKCVCPPQWTGKTCQLDANECEAKPCVNARSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSESGGKFTCDCNKGFTGTYCHENINDCEGNPCTNGGTCIDGVNSYKCICSDGWEGAHCENNINDCSQNPCHYGGTCRDLVNDFYCDCKNGWKGKTCHSRDSQCDEATCNNGGTCYDEVDTFKCMCPGGWEGTTCNIARNSSCLPNPCHNGGTCVVNGDSFTCVCKEGWEGPICTQNTNDCSPHPCYNSGTCVDGDNWYRCECAPGFAGPDCRININECQSSPCAFGATCVDEINGYQCICPPGHSGAKCHEVSGRSCITMGRVILDGAKWDDDCNTCQCLNGRVACSKVWCGPRPCRLHKGHGECPNGQSCIPVLDDQCFVRPCTGAGECRSSSLQPVKTKCTSDSYYQDNCANITFTFNKEMMSPGLTTEHICSELRNLNILKNVSAEYSIYIACEPSLSANNEIHVAISAEDIRDDGNPVKEITDKIIDLVSKRDGNSSLIAAVAEVRVQRRPLKNRTDFLVPLLSSVLTVAWVCCLVTAFYWCVRKRRRKPSSHTHSAPEDNTTNNVREQLNQIKNPIEKHGANTVPIKDYENKNSKMSKIRTHNSEVEEDDMDKHQQKVRFAKQPVYTLVDREEKVPQRTPTKHPNWTNKQDNRDLESAQSLNRMEYIV

SEQ ID NO: 4 - protein, 1219 aa, Artificial Sequence; identical to SEQ ID NO: 3
  Description of Artificial Sequence Synthetic polypeptide

SEQ ID NO: 5 - protein, 1218 aa, Artificial Sequence
  Description of Artificial Sequence Synthetic polypeptide
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

(19) United States
(12) Patent Application Publication
(10) Pub. No.: US 2015/0352180 A1
(43) Pub. Date: Dec. 10, 2015

(54) Notch Ligand Bound Biocompatible Substrates And Their Use In Bone Formation

(71) Applicant: THE TRUSTEES OF THE UNIVERSITY OF PENNSYLVANIA, Philadelphia, PA (US)

(72) Inventors: KURT DAVID HANKENSON, PHILADELPHIA, PA (US); MICHAEL IRVING DISHOWITZ, PHILADELPHIA, PA (US); JASON ALAN BURDICK, PHILADELPHIA, PA (US); FENGCHANG ZHU, PHILADELPHIA, PA (US); JAIMO AHN, PHILADELPHIA, PA (US)

(73) Assignee: The Trustee of the University of Pennsylvania, Philadelphia, PA (US)

(21) Appl. No.: 14/760,371

(22) PCT Filed: Jan. 10, 2014
(86) PCT No.: PCT/US14/11018

§ 371 Date: Jul. 10, 2015

Related U.S. Application Data
(60) Provisional application No. 61/751,676, filed on Jan. 11, 2013.
(60) Provisional application No. 61/815,808, filed on Apr. 25, 2013.

(51) Int. Cl. A61K 38/17; A61K 47/42; A61K 47/02; A61K 47/48; A61K 35/28

(52) U.S. Cl. CPC A61K 38/177; A61K 47/48192; A61K 35/28; A61K 47/02; A61K 47/42; C07K 2319/30

(57) ABSTRACT

Disclosed herein are compositions comprising an osteoinductive Notch ligand bound to at least one biocompatible substrate. Also disclosed are methods of treating patients in need of bone tissue formation through the administration of a composition comprising an osteoinductive Notch ligand bound to at least one biocompatible substrate. Further provided are methods of treating a patient in need of bone tissue formation comprising administering to said patient a composition comprising a nucleic acid molecule encoding a notch intracellular domain (NICD). Also disclosed are kits for promoting bone tissue formation.

### CROSS REFERENCE TO RELATED APPLICATIONS

This application claims the benefit under 35 U.S.C. §119(e) to U.S. Provisional Application No. 61/751,676, filed Jan. 11, 2013, and U.S. Provisional Application No. 61/815,808, filed Apr. 25, 2013. The contents of each of these applications are incorporated by reference herein in their entirety.

### GOVERNMENT RIGHTS

The subject matter disclosed herein was made with government support under the Ruth L. Kirschstein National Research Service Award (NIH/NIDCR F31DE020231), the Congressionally Directed Medical Research Program from the Department of Defense (W81XWH-10-1-0825), the Osteosynthesis Trauma and Care Foundation, the Penn Center for Musculoskeletal Disorders (NIH/NIAMS P30 AR050950), and NIH/NIA R03 AG040670. The Government has certain rights in the herein disclosed subject matter.

### SEQUENCE LISTING

The instant application contains a Sequence Listing which has been submitted electronically in ASCII format and is hereby incorporated by reference in its entirety. Said ASCII copy, created on Jan. 8, 2014, is named UPN-5914-14-6914_SL.txt and is 53,096 bytes in size.

### TECHNICAL FIELD

Provided herein are implantable compositions comprising an osteoinductive Notch ligand bound to at least one biomaterial substrate and methods of treatment using the same. Also provided herein are methods of treatment using compositions comprising a Notch Intracellular Domain.

### BACKGROUND

A significant proportion of bone injuries, including fractures, fails to produce viable callus tissue, resulting in delayed or non-union healing. Current therapies have significant limitations and a clinical need persists for the development of new methods to promote bone tissue formation. As well, there is a desire to more rapidly heal bone fractures that heal in an uncompromised manner.

With age, bone becomes more sensitive to force and is therefore more prone to fractures, which are prone to heal more slowly and develop into mal-unions at a higher rate. Age also comes with comorbidities that increase the risk of falling. Apart from the naturally occurring decrease of bone density that comes with aging, osteoporosis plays a major role in the amount of fractures from which the elderly suffer. These fractures do not heal as well as fractures in younger people. The elderly therefore suffer greatly from fractures. Looking at hip fractures, a typical geriatric and osteoporosis-related fracture, mortality and the morbidity are high, even in the long run. It is therefore important to develop new therapeutics to increase the robustness and speed of geriatric fracture healing.

Thus, there is a need for compositions to promote bone tissue formation and methods of treatment using the same.

### SUMMARY

Provided herein are implantable compositions comprising an osteoinductive Notch ligand bound to at least one biocompatible substrate.

Also provided are methods of treating a patient in need of bone tissue formation comprising administering a composition comprising an osteoinductive Notch ligand bound to at least one biocompatible substrate to a patient in need of treatment.

Methods of treating a patient in need of bone tissue formation by administering a composition comprising a nucleic acid molecule encoding a notch intracellular domain (NICD) are further disclosed.

Further disclosed are kits comprising a pharmaceutically acceptable package comprising an osteoinductive Notch ligand and at least one biocompatible substrate.

The general description and the following detailed description are exemplary and explanatory only and are not restrictive of the disclosed compositions, kits, and methods as defined in the appended claims. Other aspects will be apparent to those skilled in the art in view of the detailed description as provided herein.

### BRIEF DESCRIPTION OF THE DRAWINGS

The summary, as well as the following detailed description, is further understood when read in conjunction with the appended drawings. For the purpose of illustration, there are shown in the drawings exemplary embodiments of the disclosed compositions, kits, and methods; however, the claimed compositions, kits, and methods are not limited to the specific methods, compositions, and devices disclosed therein. In addition, the drawings are not necessarily drawn to scale. In the drawings:

FIG. 1, is a bar graph depicting gene expression of the Notch target gene Hey1 in human mesenchymal stem cells (hMSCs) grown on Jagged-1 bound to a polymer comprised of diethylene glycol diacrylate and isobutylamine (referred to herein as “A6”). Gene expression was analyzed via quantitative real time polymerase chain reaction (qPCR). Solid lines indicate significance (p<0.050) and dashes lines indicate a trend (p<0.100) between Jagged-1 doses (0, 2.5, 10 μg/mL) for direct or indirect strategies. A common letter (a,b) above any two bars indicates significance (p<0.050) between direct and indirect strategies at that given Jagged-1 dose.

FIG. 2, comprising FIGS. 2A-B, is a bar graph (FIG. 2A) depicting the amount of Jagged-1 successfully bound to the A6 polymer comprised of diethylene glycol diacrylate and isobutylamine during indirect and direct immobilization strategies, and a line graph (FIG. 2B) depicting the amount of Jagged-1 released over time from the A6 polymer comprised of diethylene glycol diacrylate and isobutylamine Protein amount was quantified using an enzyme-linked immunosorbent assay (ELISA). Solid lines indicate significance (p<0.050) between Jagged-1 doses for direct or indirect strategies. A common letter (a,b) above any two bars indicates significance (p<0.050) between direct and indirect strategies at that given Jagged-1 dose.

FIG. 3, comprising FIGS. 3A-B, is a bar graph depicting viability of human mesenchymal stem cells (hMSCs) cultured on Jagged-1 immobilized to the A6 polymer comprised of diethylene glycol diacrylate and isobutylamine in standard growth media (FIG. 3A) and osteogenic media (FIG. 3B). Cell number was assessed using an alamar Blue assay. Solid line indicates significance (p<0.050).

FIG. 4, comprising FIGS. 4A-B, is a bar graph depicting gene expression of the osteoblast differentiation markers bone sialoprotein (BSP) (FIG. 4A) and alkaline phosphatase (FIG. 4B) in human mesenchymal stem cells (hMSCs) cultured on Jagged-1 bound to the A6 polymer comprised of diethylene glycol diacrylate and isobutylamine in standard growth media. Gene expression was assessed via qPCR. Solid lines indicate significance (p<0.050) and dashes lines indicate a trend (p<0.100) between Jagged-1 doses for direct or indirect strategies. A common letter (a,b) above any two bars indicates significance (p<0.050) and a common symbol (#) indicates a trend (p<0.100) between direct and indirect strategies at that given Jagged-1 dose.

FIG. 5, comprising FIGS. 5A-C, is an image of alkaline phosphatase (AP) enzymatic activity in human mesenchymal stem cells (hMSCs) cultured on Jagged-1 bound to the A6 polymer comprised of diethylene glycol diacrylate and isobutylamine in standard growth media (FIG. 5A), and bar graphs quantifying the area of activity (FIGS. 5B and 5C). AP enzymatic activity was assessed using an AP staining kit. Solid lines indicate significance (p<0.050) and dashes lines indicate a trend (p<0.100) between Jagged-1 doses for direct or indirect strategies. A common letter (a,b) above any two bars indicates significance (p<0.050) between direct and indirect strategies at that given Jagged-1 dose.

FIG. 6, comprising FIGS. 6A-C, is a bar graph quantifying the amount of calcified mineral deposition of human mesenchymal stem cells (hMSCs), indicative of terminal osteoblast differentiation, cultured on Jagged-1 bound to the A6 polymer comprised of diethylene glycol diacrylate and isobutylamine in osteogenic media (FIG. 6A), with a corresponding image depicting of the average amount of mineral deposition (FIG. 6B), and an image depicting the average amount of mineral deposition of hMSCs cultured on Jagged-1 bound to tissue culture plastic (TCPS) (FIG. 6C). Calcified mineral was stained using Alizarin Red S. Areas of dense mineral appear black. A6 polymer stains yellow. TCPS does not stain. hMSCs were cultured in osteogenic media. Scale bars are 1 mm.

FIG. 7, comprising FIGS A-B, depicts trabecular bone formation in wild type (WT) mice with endogenous Jagged-1 compared to mice with Jagged-1 conditionally deleted in mesenchymal progenitor cells (FIG. 7A) or committed osteoblasts (FIG. 7B). Mice with Jagged-1 conditionally deleted in mesenchymal progenitor cells (A: Prx1-Cre Jag1^{f/f}) or committed osteoblasts (B: Col2.3-Cre Jag1^{f/f}). * p<0.050, # p<0.100.

FIG. 8 depicts osteoblast activity (measured by Alkaline Phosphatase gene expression) relative to Jagged-1 gene expression.

FIG. 9 depicts in vivo human mesenchymal stem cell (hMSC) mediated bone formation, measured by micro-computed tomography, following implantation of Jagged-1-scaffold-hMSC. * p<0.05.

FIG. 10 depicts an increase in bone regeneration following direct delivery of recombinant rat Jagged-1 on a resorbable collagen matrix.

FIG. 11, comprising FIGS. 11A and B, depicts osteoblastogenesis in hMSCs that were plated onto surface-bound Jagged-1 (FIG. 11A) or treated with soluble Jagged-1 (FIG. 11B). Cells were harvested at day 4 for alkaline phosphatase staining or day 12 for Alizarin Red S (calcium mineral).

FIG. 12, comprising FIGS. 12A-G, depicts osteoblastogenesis in the areas where Jagged-1 (Jag1) is bound to the TC plates. (A) Scanned 6-well plate. (B/C) Jag1 or (D/E) controls (Anti-Fc Ab or Anti-FC Ab+BSA) were spotted in the center of wells and then hMSC plated to the wells for 6 days. Osteoblast induction was determined using Alk Phos staining as in FIG. 11. (G) BMP2 as a positive control induces AP throughout the plate. Very little AP staining was present in control wells (D-F).

FIG. 13, comprising FIGS. 13A-13D, depicts a histological analysis of cartilage in NICD treated fractures. NICD is shown in red (left bars in each pair); GFP is shown in yellow (right bar in each pair). A) DPF 10 cartilage percentage—young NICD mice show higher percentages of cartilage. B) DPF 10 cartilage area—both young and old NICD mice have a larger cartilaginous area. C) DPF 20 cartilage percentage—young NICD mice have a lower percentage of cartilage, supporting microCT data that show increased calcification in these mice. D) DPT 20 cartilage area—like C, the cartilage area is lower in young NICD mice, again supporting the increased calcification in these tibiae.

FIG. 14, comprising FIGS. 14A-14C, depict a histological analysis of the total callus area in NICD treated fractures. NICD is shown in red (left bars in each pair); GFP is shown in yellow (right bar in each pair). A) Total callus area DPF 10—both the young and old NICD mice show increased callus area, corresponding with the microCT data. B) Total callus area DPF 20—interestingly, the total callus are in the young NICD tibiae is smaller than the GFP tibiae, which contradicts the microCT data. As expected, the old NICD tibiae do have a larger callus area. C) Total callus area DPF 40—corresponding with the microCT data, both young and old NICD tibiae have a larger callus area.

FIG. 15, comprising FIGS. 15A-15D, depict a micoCT (μCT) analysis of NICD treated fractures. NICD is shown in red (left bars in each pair); GFP is shown in yellow (right bar in each pair). A) DPF 20 total callus volume—shows significant difference in both young and old mice (p=0.0497 and p=0.028, respectively). B) DPF 40 bone volume fraction—shows significant difference in old mice (p=0.030). C) DPF 40 trabecular thickness—shows significant difference in old mice (p=0.038). D) DPF 40 tissue mineral density—shows significant difference in young mice (p=0.01907).

FIG. 16, comprising FIGS. 16A-6B, depicts exemplary histology samples stained with Fast Green and Safranin-O in (A) 5 month old NICD vs. GFP tibia at 10, 20, and 40 DPF and (B) 25 month old vs. GFP tibia at 10, 20, and 40 DPF.

FIG. 17, comprising FIGS. 17A-17B, depicts exemplary microCT samples showing both transversal cuts and 3D reconstruction in (A) 5 month old NICD vs. GFP tibia at 10, 20, and 40 DPF and (B) 25 month old vs. GFP tibia at 10, 20, and 40 DPF.

FIG. 18, comprising FIGS. 18A-18B, depict human or mouse MSC cells transfected with NICD1, NICD2, or control viral supernatants and (A) stained for alkaline phosphatase activity (ALP) at day 5 (left panel, purple) and stained with Alizarin Red S at day 10 for calcium mineral (right panel, red) or (B) qRT-PCR analysis of alkaline phosphatase (left panel) or Hey1 (right panel) expression.

### DETAILED DESCRIPTION OF ILLUSTRATIVE EMBODIMENTS

The disclosed compositions, kits, and methods may be understood more readily by reference to the following detailed description taken in connection with the accompanying figures and examples, which form a part of this disclosure. It is to be understood that the disclosed compositions, kits, and methods are not limited to the specific composition, methods, kits, applications, conditions or parameters described and/or shown herein, and that the terminology used herein is for the purpose of describing particular embodiments by way of example only and is not intended to be limiting of the claimed compositions, kits, and methods. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the disclosed compositions, kits, and methods which are, for clarity, described herein in the context of separate embodiments, may also be provided in combination in a single embodiment. Conversely, various features of the disclosed compositions, kits, and methods that are, for brevity, described in the context of a single embodiment, may also be provided separately or in any subcombination. Further, reference to values stated in ranges include each and every value within that range.

Provided herein are implantable compositions comprising an osteoinductive Notch ligand bound to at least one biocompatible substrate.

As used herein, the term “osteoinduction” refers to the ability to induce or stimulate bone formation. As used herein, the term “osteoinductive” refers to a material with the ability to induce or stimulate bone formation. As used herein, the term “osteogenesis” refers to the formation of bone. At times, the terms used in the field to mean promotion or induction of bone formation are used inconsistently. Therefore, any term used in the art relating to promotion or induction of bone formation can be used interchangeably herein.

As used herein, the term “biocompatible” refers to the ability to interface with biological systems without toxic or injurious effects. Thus, a “biocompatible substrate” is a material that can be used to replace or function together with a biological system or living tissue, performing an appropriate host response in a specific situation. Biocompatible substrates for use in the disclosed invention can be synthetic, natural, or any combination thereof.

One with skill in the art would know that multiple types of biocompatible substrates exist. Biocompatible substrates of the present invention include, but are not limited to, calcium and phosphate-based ceramics, allografts, autografts, polymers, bioglass, collagen-based materials, proteoglycan-based materials, glycosaminoglycan-based materials, polysaccharides, biomaterials that are clinically used for musculoskeletal applications, and any combination thereof.

The biocompatible substrates used in the present invention can be present in various forms. These include, but are not limited to, solid scaffolds, aqueous solutions, gels, hydrogels, beads, microbeads, nanoparticles, or some other delivery vehicle useful for delivery of a Notch ligand.

As used herein, the term “implantable” refers to the ability to be administered into a patient, through implantation, injection, or some other form of administration. Thus, the compositions of the present invention can be in any form suitable for administration into a patient. Such compositions also possess characteristics allowing them to be placed within a patient, such as sterile.

Thus, in some embodiments, the biocompatible substrate can be a calcium and phosphate-based ceramic. Calcium and phosphate-based ceramics include, but are not limited to, hydroxyapatite and β-tricalcium phosphate. In other embodiments, the biomaterial can be allografts, including but not limited to demineralized bone matrix. In other embodiments, the biomaterial can be autografts. Autografts for use in the present invention include, but are not limited to, autologous bone grafts harvested from the patient's iliac crest. In certain embodiments, the biomaterial can be a polymer. Polymers for use in the present invention include, but are not limited to, polylactic acid, polyglycolic acid, poly(D/L-lactic co-glycolic) acid, poly-e-caprolactone, and poly(β-amino ester)s. For example, in some aspects, the poly(β-amino ester)s comprise diethylene glycol diacrylate and isobutylamine (referred to herein as “A6”). In other embodiments, the biomaterial can be a bioglass. Bioglasses for use in the present invention include, but are not limited to, those composed of SiO₂, Na₂O, CaO, and P₂O₅, such as 45S5 and 5S4.3. In certain embodiments, the biomaterial can comprise collagen-based materials. Collagen-based materials for use in the present invention include, but are not limited to, mammalian type I collagen, rat tail tendon collagen, gelatin, or any combination thereof. For example, the biocompatible substrate can comprise a resorbable collagen matrix. In other embodiments, the biomaterial can be proteoglycan- or glycosaminoglycan-based materials such as hyaluronic acid. In other embodiments, the biomaterial can be a polysaccharide, including but not limited to chitosan, chitin, and cellulose.

The biomaterials can be composed of a single biomaterial or composed of combinations of multiple biomaterials. Thus, in some embodiments, the biomaterial is composed of a single calcium and phosphate-based ceramic, allograft, autograft, polymer, bioglass, collagen-based material, proteoglycan-based material, glycosaminoglycan-based material, or polysaccharide. In other embodiments, the biomaterial comprises any combination of calcium and phosphate-based ceramics, allografts, autografts, polymers, bioglass, collagen-based materials, proteoglycan-based materials, glycosaminoglycan-based materials, and polysaccharides. Combinations of biomaterials include mixtures of the same biomaterial type and mixtures of multiple biomaterial types. For example, and without intent to be limiting, in some embodiments the biomaterial can comprise a mixture of polymers. In other embodiments, the biomaterial can comprise a mixture of polymers together with another biomaterial such as polysaccharides.

In certain embodiments, the biocompatible substrate can comprise one or more poly(β-amino esters) (PBAE). As used herein, the term “poly(β-amino ester)” refers to a biomaterial substrate that is formed by a condensation polymerization of a diacrylate and an amine. Such condensation polymerizations can be photocrosslinkable. Some suitable poly(β-amino ester)s include those described in Anderson et al., Advanced Biomaterials (2006) 18:2614-2618 and in U.S. Pat. App. No. US20080145338 by Anderson et al., entitled “Crosslinked, Degradable Polymers and Uses Thereof” For example, in some aspects the biocompatible substrate can comprise “A6,” formed from a condensation polymerization of diethylene glycol diacrylate and isobutylamine.

The ratio of diacrylate to amine is based in part on the specific combinations used. A suitable ratio of diacrylate to amine can be 2 to 0.2, more preferably 1.8 to 0.4, more preferably 1.6 to 0.6, and more preferably 1.4 to 0.8, and even more preferably 1.2 to 1.0.

In some embodiments, the biocompatible substrate is biodegradable. As used herein, the term “biodegradable” refers to the ability to degrade or break down inside the body. Thus, “biodegradable material” refers to biocompatible substrates that degrade or break down inside the body of a human or non-human subject.

In other embodiments, the biocompatible substrate is non-biodegradable. As used herein, the term “non-biodegradable” refers to a biocompatible or bioinert material that is permanent or slow degrading within the body. Thus, “non-biodegradable material” refers to biocompatible substrates that are permanent or slow degrading within the body of a human or non-human subject.

As used herein, the term “Notch ligand” refers to a protein or peptide that binds to a Notch receptor and activates a Notch signaling pathway. The Notch ligand used in the present invention can be derived from any mammalian species, and includes human and non-human Notch ligands. Preferably, the Notch ligand is capable of activating a human notch receptor, including Notch1, Notch2, Notch3, Notch4, or any combination thereof. Notch ligands include Delta-like-ligands (Dll) and Jagged ligands. In mammals, multiple Dll and Jagged ligands exist.

Preferably, the Notch ligand is capable of activating a Notch receptor present on a cell that has osteogenic potential.

In a preferred aspect, the Notch ligand can be a Jagged-1 protein. Jagged-1 used in the present invention can be derived from any mammalian species. Thus, in some embodiments, the Jagged-1 protein can be a human Jagged-1 protein. In other embodiments, the Jagged-1 protein can be a non-human Jagged-1 protein. For example, in certain embodiments Jagged-1 can be a mouse Jagged-1. In other embodiments, Jagged-1 can be a rat Jagged-1.

Jagged-1 serves as a ligand for multiple Notch receptors and is involved in the regulation of Notch signaling. Jagged-1 naturally occurs as a transmembrane protein containing extracellular, transmembrane, and intracellular regions. The extracellular region of Jagged-1 contains, in order from the N-terminus to the transmembrane domain: an N-terminal domain; a DSL domain; a series of EGF-like repeats; and a cysteine-rich region (CR domain). The intracellular region of Jagged-1 contains the C-terminal domain. Multiple regions of the Jagged-1 extracellular domain are involved in binding to the Notch receptor, including the DSL and EGF-like repeats. Upon binding of Jagged-1 to a Notch receptor, the intracellular domain of the Notch receptor (herein referred to as “Notch Intracellular Domain” or “NICD”) is cleaved off, which subsequently translocates into the nucleus where it binds to co-activators to initiate transcription of Notch target genes. This signaling cascade is referred to as canonical Notch signaling. Ligand binding may also signal in a non-canonical manner.

Activation of the Notch receptor can be transient or sustained. In some embodiments, binding of Jagged-1 to the Notch receptor results in the transient activation of the receptor. In other embodiments, binding of Jagged-1 to the Notch receptor results in the sustained activation of the receptor. Transient signaling refers to activation of canonical Notch signaling for discrete time periods (1-3 days), whereas sustained Notch signaling refers to Notch signaling being activated for longer periods of time.

In some aspects, the Jagged-1 protein can be full length native mammalian Jagged-1. Thus, Jagged-1 in accordance with the present invention can have an amino acid sequence that is substantially similar to the native mammalian Jagged-1 amino acid sequence. For example, in certain embodiments, Jagged-1 can have an amino acid sequence substantially identical to SEQ ID NO:1. SEQ ID NO:1 is the amino acid sequence for human Jagged-1 and is identified by GenBank Accession No. AAC51731. SEQ ID NO:2 is the amino acid sequence for mouse Jagged-1 and is identified by GenBank Accession No. AAF15505. SEQ ID NO:3 is the amino acid sequence for rat Jagged-1 and is identified by GenBank Accession No. NP_{—}062020.

In certain embodiments, Jagged-1 comprises a portion of full length Jagged-1, such as a fragment of Jagged-1, which is shorter than full length Jagged-1 but which maintains the ability to bind to and activate a Notch receptor. For example, in some embodiments, the Jagged-1 fragment comprises the DSL domain. Thus, in some aspects, the Jagged-1 fragment comprises amino acids 185-229 of SEQ ID NO:1. In other aspects, the Jagged-1 fragment comprises amino acids 185-229 of SEQ ID NO:2. In other aspects, the Jagged-1 fragment comprises amino acids 185-229 of SEQ ID NO:3. In other embodiments, the Jagged-1 fragment comprises the DSL and one or more EGF-like repeat domains. In yet other embodiments, the Jagged-1 fragment comprises the DSL and one or more EGF-like domains together with other portions of the extracellular region of Jagged-1.

In other embodiments, the fragment of Jagged-1 is a variant that mimics the action of a fragment containing the DSL and EGF-like repeat domains. In other embodiments, the fragment comprises those other portions of Jagged-1 that are required to activate cleavage of the Notch receptor intracellular domain and initiate downstream Notch signaling.

Fragments of Jagged-1 for use in the disclosed compositions include naturally occurring fragments and synthetic peptides that contain the necessary regions to bind to and activate Notch receptor signaling.

In other embodiments, full length Jagged-1 or a fragment thereof can be a variant of native mammalian Jagged-1. Variants of Jagged-1 include, but are not limited to, recombinant Jagged-1, a Jagged-1 fusion protein, an affinity tagged Jagged-1, a fluorescent marker tagged Jagged-1, a chimeric Jagged-1, or any combination thereof.

In other embodiments, full length Jagged-1 or a fragment thereof can be an analog of Jagged-1, a derivative of Jagged-1, a peptide sequence of Jagged-1, or any combination thereof. Thus, in some embodiments, Jagged-1 comprises a polypeptide encoded by a naturally occurring allelic variant of native Jagged-1 gene. In other embodiments, Jagged-1 comprises a polypeptide encoded by an alternative splice form of a native Jagged-1 gene. In yet other embodiments, the Jagged-1 comprises a polypeptide encoded by a homolog or ortholog of a native Jagged-1 gene. In other embodiments, the Jagged-1 comprises a polypeptide encoded by a non-naturally occurring variant of a native Jagged-1 gene.

Variants of Jagged-1 for use in the disclosed composition include those protein or peptide sequences that differ from native Jagged-1 polypeptide in one or more amino acids. The peptide sequence of such variants can feature a deletion, addition, or substitution of one or more amino acids of a native Jagged-1. Amino acid insertions can be from about 1 to 4 contiguous amino acids, and deletions can be from about 1 to 10 contiguous amino acids; however, differing lengths of insertions or deletions may be used to modify Jagged-1. Variant Jagged-1 polypeptides substantially maintain a native Jagged-1 functional activity. Jagged-1 polypeptide variants can also feature silent or conservative amino acid substitutions.

Suitable proteins and peptides can include those that are at least 50%, 60%, 70%, 80%, 85%, 90%, 91%. 92%, 93%, 94%, 95%, 96%, 97%, 98%, or 99% identical to the amino acid sequence of SEQ ID NO:1, SEQ ID NO:2, or SEQ ID NO:3.

Jagged-1 variants can include agonistic forms of the protein that constitutively express the functional activities of native Jagged-1. Other Jagged-1 variants can include those that are resistant to proteolytic cleavage, as for example, due to mutations, which alter protease target sequences. Whether a change in the amino acid sequence of a peptide results in a variant having one or more functional activities of a native Jagged-1 can be readily determined by testing the variant for a native Jagged-1 functional activity.

Recombinant Jagged-1 includes mammalian Jagged-1 protein expressed in bacteria or mammalian cells following introduction of a vector or plasmid containing a nucleic acid encoding Jagged-1 or a variant of Jagged-1 into the cells. Recombinant Jagged-1 proteins can be substantially similar in amino acid sequence to the naturally occurring Jagged-1, or can differ from the naturally occurring Jagged-1 at one or more amino acid residues.

Fusion or tagged proteins include full length Jagged-1 or Jagged-1 variants containing additional domain sites for added benefit. Addition of a tag allows the protein to be distinguished from endogenous proteins. For example, in some embodiments, Jagged-1 can be fused to an immunoglobulin Fc region. The immunoglobulin Fc region can comprise IgG, IgM, IgA, IgD, IgE, or any combination thereof. Various immunoglobulin subclasses can also be fused to the Notch ligand including, but not limited to, IgG₁, IgG₂, IgG₃, IgG₄, or any combination thereof. SEQ ID NO:4 is the amino acid sequence for rat Jagged-1 fused to human Fc IgG₁. SEQ ID NO:5 is the amino acid sequence for human Jagged-1 fused to human Fc IgG₁.

In other embodiments, Jagged-1 can be tagged with an affinity tag. One with skill in the art would know that multiple affinity tags exist for protein applications. Affinity tags include, but are not limited to, His, FLAG, HA, Calmodulin, Myc, S, SBP, and AviTag.

In other embodiments, Jagged-1 can be tagged or fused to a fluorescent marker. One with skill in the art would know that multiple fluorescent tags exist for protein applications. These include, but are not limited to, CFP, GFP, YFP, OFP, and RFP.

In some embodiments, Jagged-1 can be a chimeric protein. A chimeric Jagged-1 can be generated by fusing a Jagged-1 from one species to a portion of a protein or tag from a different species. For example, in some embodiments, recombinant rat Jagged-1 can be fused to a human Fc region, creating a chimeric protein. SEQ ID NO:4 is the amino acid sequence for rat Jagged-1 fused to human Fc IgG₁.

The above description of Jagged-1 proteins is equally applicable to other Notch ligands. Therefore, the composition can contain any osteoinductive Notch ligand present as a full length protein, fragment, or variant, such as described above for Jagged-1.

The amount of Notch ligand bound to the biocompatible substrate can vary. The Notch ligands of the disclosed compositions will generally be used in an amount effective to achieve the intended purpose. For use in bone tissue formation, Notch ligands will be present in a therapeutically effective amount capable of promoting bone tissue formation.

In some embodiments, immobilization of Jagged-1 to the biocompatible substrate allows for the ligand to apply the required tension on the Notch receptor after binding to the receptor. This tension can promote the series of cleavage events responsible for releasing intracellular domain of the Notch receptor, which triggers downstream activation of the Notch signaling pathway.

In various aspects, the Notch ligand is sufficiently present to induce activation and downstream signaling of a Notch receptor. As used herein, “sufficiently present” refers to an effective amount of Jagged-1 to achieve the intended purpose. A suitable amount of Jagged-1 protein can be those amounts that fully or partially saturate the biocompatible substrate.

In some embodiments, the Notch ligand can be present in an amount capable of inducing expression of Notch target genes. For example, in some aspects, the Notch ligand induces expression of Hey1. In other aspects, the Notch ligand induces expression of Hes1. In other embodiments, the Notch ligand induces the expression of other Notch target genes.

In some embodiments, the Notch ligand can be present in an amount capable of increasing the gene expression of osteogenic differentiation markers. For example, in some aspects of the invention, the Notch ligand can increase the expression of the osteogenic differentiation marker bone sialoprotein. In other aspects of the invention, the Notch ligand can increase the expression of the osteogenic differentiation marker alkaline phosphatase. In other aspects of the invention, the Notch ligand can increase the expression of yet other osteogenic differentiation markers. In other aspects of the invention, the Notch ligand can increase the expression of multiple osteogenic markers including bone sialoprotein, alkaline phosphatase, other genes expressed during osteoblast differentiation, or any combination thereof.

In some embodiments, the Notch ligand can be present in an amount capable of increasing the activity of enzymes characteristic of osteoblast differentiation and bone tissue formation. For example, in one aspect of the invention, the Notch ligand can increase the activity of alkaline phosphatase.

In other embodiments, the Notch ligand can be present in an amount capable of leading to the production of a hydroxyapatite mineralized matrix, which comprises the major inorganic component of bone.

Suitable ranges of Notch ligand bound to the biocompatible substrate include, for example, 1 microgram (μg) to 100 mg, preferably 50 μg to 50 mg, and more preferably 1 mg to 10 mg.

The Notch ligand can be directly or indirectly bound to the biomaterial substrate. In a preferred aspect, the Notch ligand can be directly bound to the substrate. Direct binding of the ligand to the biocompatible substrate can be accomplished through a variety of techniques known in the art. For example, the Notch ligand can be covalently bound to the biocompatible substrate. In other embodiments, the Notch ligand can be bound to the biocompatible substrate through non-covalent adsorption. Absorption of the Notch ligand occurs naturally without introduction of any additional chemical or physical factor. In one embodiment, absorption of the Notch ligand can occur by dissolving the ligand in a buffer, including but not limited to saline, and adding it directly to the biomaterial substrate, such that over time the ligand becomes bound to the substrate. Absorption of the Notch ligand to the substrate can occur randomly. Thus, in some embodiments, the Notch ligands can be bound in different orientations. In other embodiments, the Notch ligands can be bound in the same orientation. In a preferred aspect, the Notch ligands are bound in an orientation that allows the ligand to interact with and activate a Notch receptor.

In other aspects, the Notch ligand can be indirectly bound to the biocompatible substrate. Indirect binding can be performed through a variety of techniques including, but not limited to, the use of antibodies, linkers, or any combination thereof.

In some embodiments, an appropriate antibody can be used to indirectly bind the Notch ligand to the substrate. The antibody can be first bound to the biocompatible substrate, followed by binding of the Notch ligand to the antibody. Alternatively, an appropriate antibody can be first bound to the Notch ligand, followed by binding the antibody-ligand complex to the biocompatible substrate. Binding of antibody to the biocompatible substrate can be performed through covalent coupling, adsorption, or other methods known in the art.

Notch ligands that are indirectly bound to the biocompatible substrate through an antibody can be bound in the same or different orientations. In some embodiments, the Notch ligands can be bound in different orientations. In other embodiments, the Notch ligand can be bound in the same orientation. In a preferred aspect, the Notch ligands are bound in an orientation that allows the ligand to interact with and activate a Notch receptor.

Antibodies can bind to the biocompatible substrate in the same or different orientations. In some embodiments, the antibody is bound to the substrate in the same orientation. In other embodiments, the antibody can be bound in different orientations.

Antibodies for use in the disclosed compositions include any antibody that binds to full length Jagged-1, a fragment of Jagged-1, or any variant, recombinant, analogue, derivative, chimeric, or any combination thereof. Such antibodies recognize and bind Jagged-1 in a manner in which Jagged-1 retains the ability to bind to and activate a Notch receptor. Thus, some antibodies bind a portion of the Jagged-1 protein that is not involved in binding to the Notch receptor. In some embodiments an anti-Jagged-1 antibody can be used to indirectly bind Jagged-1 to the biocompatible substrate. In other embodiments, an anti-Fc antibody can be used to indirectly bind a Jagged-1 protein fused with an Fc portion to the biocompatible substrate. In other embodiments, an antibody directed toward a tag present on Jagged-1 can be used. For example, an anti-His antibody can be used to indirectly bind a His-tagged Jagged-1 protein to the biocompatible substrate.

Linkers include any protein, peptide, or molecule, whether natural or synthetic, which can be used to bind a Notch ligand to the biocompatible substrate.

The Notch ligand bound substrate can be delivered to the site in need of bone tissue formation. One with skill in the art would know that bone tissue formation and repair can occur through various mechanisms, including endochondral ossification, intramembranous ossification, or a combination thereof. As used herein, the term “endochondral ossification” refers to the formation of bone tissue in which a cartilage template is first produced and then replaced by bone tissue. The terms “endochondral ossification” and “endochondral bone regeneration” are used interchangeably herein. As used herein, the term “intramembranous ossification” refers to direct bone tissue formation which occurs without a cartilage precursor. The terms “intramembranous ossification” and “intramembranous bone regeneration” are used interchangeably herein.

In some embodiments, the composition further comprises cells. The cells can attach to the biocompatible substrate and interact with the Notch ligand. In a preferred aspect, the biocompatible substrate can serve as a scaffold by supporting cell adhesion. The Notch ligand can then interact with Notch receptors expressed by these cells and induce osteoblast differentiation and bone tissue formation.

It would be known by one with skill in the art that numerous bonds are involved in protein-protein interactions, such as those between the Notch ligand and the Notch receptor. These bonds include, but are not limited to, covalent bonds, hydrogen bonds, electrostatic interactions, hydrophobic and hydrophilic interactions, Van der Waals forces, and any combination thereof.

Cells for use in the disclosed compositions include, but are not limited to, adult stem cells, osteogenic cells, bone marrow aspirate, embryonic stem cells, induced pluripotent stem cells, or any combination thereof.

Thus, in some embodiments, the composition can include mesenchymal stem cells. As used herein, the term “mesenchymal stem cells” (MSCs), also known as “mesenchymal stromal cells,” refers to a population of multipotent adult stem cells from mesenchymal origin that have the ability to differentiate into, or has already begun to differentiate into, cells of the mesenchymal lineage. Cells of the mesenchymal lineage include osteoblasts, chondrocytes, adipocytes, fibroblasts, and myocytes. In a preferred embodiment, MSCs can interact with the Notch ligand and undergo osteoblast differentiation. MSCs can be harvested from bone marrow, adipose tissue, periosteal tissue, muscle tissue, and other tissues of mesenchymal origin.

The composition can include osteogenic cells. As used herein, the term “osteogenic cell” refers to a population of cells in the osteogenic lineage at any or multiple stages of differentiation, including MSCs, osteoprogenitor cells, immature osteoblasts, osteoblasts, osteocytes, or any combination thereof. Osteogenic cells can be harvested from a variety of sources, including but not limited to, bone tissue, or can be derived from harvested and purified MSC.

In other embodiments, the composition can include bone marrow aspirate. As used herein, the term “bone marrow aspirate” refers to a heterogeneous population of cells and tissues derived from bone marrow aspirate, including hematopoietic cells and tissues, and mesenchymal cells, specifically MSCs, and tissues. In some embodiments, bone marrow aspirate can be used. Thus, for example, a heterogenous population of cells including hematopoietic and mesenchymal cells can be used. In other embodiments, cells isolated from bone marrow aspirate can be used. For example, a homogenous population of mesenchymal cells can be isolated and used.

In other embodiments, the composition can include embryonic stem cells. As used herein, the term “embryonic stem cells” refers to a population of totipotent cells with the ability to differentiate into any cell type. These embryonic stem cells can be further differentiated to become mesenchymal stem cells or osteogenic cells. Stem cells can be harvested from a variety of sources, including but not limited to, bone marrow, adipose tissue, periosteal tissue, muscle tissue, umbilical cord, blood, or any combination thereof.

In other embodiments, the composition can include induced pluripotent stem cells (iPS cells). As used herein, the term “induced pluripotent stem cells” refers to somatic cells that have been reprogrammed to become embryonic-like stem cells. These iPS cells can be further differentiated to become mesenchymal stem cells or osteogenic cells.

If desired, cells can also be expanded ex vivo for a desired period of time prior to being attached to the biocompatible substrate Notch ligand composition. Expansion can occur until the composition is partially or fully saturated with the desired type of cell.

Also disclosed herein are methods of treating a patient in need of bone tissue formation comprising administering a composition comprising an osteoinductive Notch ligand bound to at least one biocompatible substrate to a patient in need of treatment.

As used herein, the term “patient” is intended to mean any mammal. Thus the disclosed methods are applicable to human and nonhuman subjects, although it is most preferably used in humans. In some embodiments, the patient treated can be a human. In other embodiments, the patient treated can be a rat. In yet other embodiments, the patient treated can be another mammal “Subject” and “patient” are used interchangeably herein.

As used herein, the terms “treat,” “treating,” and “treatment” include and encompass reducing, ameliorating, alleviating, reversing, inhibiting, preventing and/or eliminating bone loss and promoting, inducing, stimulating and/or supporting bone repair or bone formation. “Treat,” “treating,” and “treatment” also refer to reduction in severity and/or frequency of symptoms, elimination of symptoms and/or underlying cause, prevention of the occurrence of symptoms and/or their underlying cause, and improvement or remediation of damage.

The disclosed methods can be used to treat multiple injuries, diseases, and/or conditions that require bone tissue formation or repair. These include, but are not limited to: fractures of the hip, vertebra, tibia, femur, humerus, radius, ulna, wrist, ankle, skull and other long, short, flat and irregular bones of the upper and lower body; spinal fusions; bone tissue removal due to tumor excision, infection, or other complications; facial reconstruction; dental reconstruction; distraction osteogenesis; joint revisions; osteolysis; osteoporosis; genetic diseases; or any combination thereof. Thus, for example, in some embodiments, the method can be used to treat Alagille Syndrome, which occurs due to mutations to Jagged-1 or the Notch2 receptor. In other embodiments, the method can be used to treat fractures and/or breaks caused by age. For example, the methods of the present invention can be used to enhance geriatric bone regeneration.

The compositions used in the disclosed methods will generally be used in an amount effective to achieve the intended purpose. For use to promote or induce bone tissue formation, the composition, or pharmaceutical compositions thereof, can be administered or applied in a therapeutically effective amount. The term “therapeutically effective amount” refers to an amount effective to treat the patient. Determination of a therapeutically effective amount is well within the capabilities of those skilled in the art, especially in light of the detailed disclosure provided herein.

Initial dosages can be estimated from in vitro and in vivo data, such as cell culture assays and animal models, using techniques that are well known in the art. One having ordinary skill in the art could readily optimize administration to humans based on animal data.

The amount of composition administered will, of course, be dependent on the subject being treated, on the subject's weight, the severity of the injury or disease, and the manner of administration, among others.

In some embodiments, the composition is administered in a bone in need of treatment or repair. In other embodiments, the composition is administered proximate to a bone in need of treatment or repair. In yet other embodiments, the composition is administered in and proximate to a bone in need of treatment or repair.

The compositions can be administered with or without cells bound. In some embodiments, cells can be expanded ex vivo on the Notch ligand bound biocompatible substrate prior to administration. In other embodiments, cells can be bound to the Notch ligand bound biocompatible substrate and the composition can be administered into a patient without ex vivo expansion of the cells. Administered compositions containing cells can interact with endogenous patient cells following administration, or can be saturated with cultured cells such that the administered composition cannot interact with patient cells.

In other embodiments, the Notch ligand bound biocompatible substrate can be administered into a patient without any cells attached. Administered compositions lacking cells can interact with and bind to endogenous patient cells following administration.

The compositions can be administered through implantation, injection, and various other procedures that are known in the art. In some embodiments, the compositions can be formulated in aqueous solutions, gels, or hydrogels, which can be administered through injection using a catheter, syringe, or other instrument, directly to the site of injury. Such formulations can solidify in situ following administration. In some aspects, the compositions can be injected into a patient with an open wound. In other aspects, the compositions can be injected into a patient lacking an open wound, such as a patient with an internal injury or disease that is not exposed through the skin. In some embodiments, the composition can be injected by approximating the location of interest. In other embodiments, the composition can be injected using an imaging device such as x-ray, CT, MRI, ultrasound, and other modalities to guide the surgeon in determining the location of interest.

In other embodiments, the compositions can be formulated as solid supports. In a preferred embodiment, the composition can be implanted as a solid directly to the area of interest. This can be performed in a patient with an open wound, created by injury, disease, or surgically.

In other embodiments, the compositions can be formulated as beads or nanoparticles. Such formulations can be administered to patients with or without open wounds, through various procedures such as implantation or injection.

The Notch ligand bound biocompatible substrate can interact with cultured or endogenous cells and induce osteoblast differentiation resulting in the formation of bone tissue. In some embodiments, the bone tissue formed as a result of administering the composition can be incorporated into or proximate to the bone in need of treatment.

The biocompatible substrate can be biodegradable or non-biodegradable. Thus, in some embodiments, the substrate degrades following administration into the patient in need of treatment. One with skill in the art would appreciate that degradation of the substrate is dependent upon various factors. Thus, for example, the rate of degradation can be influenced by the components of the substrate, its biocompatibility, physical and chemical structure, implant location, and the amount of time cultured prior to administration. In some aspects, the composition can degrade within six months following administration. In other aspects, the composition can degrade within twelve months following administration. In other aspects of the invention, the composition can degrade after twelve months following administration.

In other embodiments, the substrate can be non-biodegradable being incorporated in or proximate to the patient's bone for an extended period of time. Such non-biodegradable substrates can remain in or proximate to the site of treatment for months, years, or can be permanent.

Also disclosed herein are methods of treating a patient in need of bone tissue formation comprising administering to said patient a composition comprising a Notch Intracellular Domain (NICD).

In some embodiments, the NICD comprises the intracellular domain of the human Notch1 receptor (hNICD1). SEQ ID NO:6 is a cDNA sequence of hNICD1 which contains a starting ATG, and which encodes the hNICD1 protein of SEQ ID NO:7. In some embodiments, the NICD comprises the intracellular domain of the human Notch2 receptor (hNICD2). SEQ ID NO:8 is a cDNA sequence of hNICD2 which contains a starting ATG, and which encodes the hNICD2 protein of SEQ ID NO:9. In some embodiments, the NICD comprises the intracellular domain of the mouse Notch1 receptor (mNICD1). SEQ ID NO:10 is a cDNA sequence of mNICD1 which contains a starting ATG, and which encodes the mNICD1 protein of SEQ ID NO:11. In yet other embodiments, the NICD comprises the intracellular domain of the mouse Notch2 receptor (mNICD2). SEQ ID NO:12 is a cDNA sequence of mNICD2 which contains a starting ATG, and which encodes the mNICD2 protein of SEQ ID NO:13.

NICD compositions can be administered to a patient by a variety of methods known in the art. For example, in some embodiments, a vector containing the NICD can be administered directly to the patient in need of treatment. A variety of expression vectors can be utilized to administer the NICD, including, but not limited to, plasmid DNA expression vectors and viral expression vectors (i.e. retroviral vectors, lentiviral vectors, adenoviral vectors, and adeno-associated viral vectors).

The NICD compositions can be administered by numerous methods, including, but not limited to, intravenous, intradermal, intraosseous, intraarticular, intradermal, intrathecal, and subcutaneous.

In some aspects of the methods, the NICD compositions can comprise vectors containing a nucleic acid sequence encoding NICD alone. In other aspects of the methods, the NICD composition can comprise vectors containing a nucleic acid sequence encoding NICD together with a tag used to evaluate the localization of the composition within the patient. For example, the vector can comprise a nucleic acid sequence encoding NICD together with a nucleic acid sequence encoding GFP.

Administration of the NICD composition can increase callus volume, bone volume fraction, trabecular thickness, or any combination thereof.

Also disclosed herein are kits comprising a pharmaceutically acceptable package comprising an osteoinductive Notch ligand and at least one biocompatible substrate.

In some embodiments of the kits, the osteoinductive Notch ligand bound biocompatible substrate can be pre-formed. For example, the kit can contain a Notch ligand pre-bound to a biocompatible substrate. Such Notch ligand pre-bound substrates can be maintained in a biologically compatible state suitable for administration into a patient. Suitable biologically compatible states include, but are not limited to, frozen or in a sterile solution.

In other embodiments, the Notch ligand and biocompatible substrate can be present as separate components such that the Notch ligand bound biocompatible substrate can be formed at or near the time of treatment. In one example, Jagged-1 can be provided in lyophilized form, diluted in a sterile, saline-based solution to a desired concentration, added directly to the biocompatible substrate, and allowed to adsorb.

The kits can be used to promote bone tissue formation and thus treat or repair bone injuries in a subject in need of treatment.

The kits include biocompatible substrates to be used in the formation of osteoinductive Notch ligand bound biocompatible substrate. Any of the biocompatible substrates or combinations thereof disclosed herein are suitable to use in the kits of the present invention.

In some embodiments, the biocompatible substrates can be in a form capable of being dissolved in a solution. In other embodiments, the biocompatible substrates can be in aqueous form.

The kits also contain Notch ligand to be bound to the biocompatible substrate. Any of the Notch ligands disclosed herein are suitable to use in the disclosed kits. In some embodiments, the Notch ligand can be in a form capable of being dissolved in a solution. In other embodiments, the Notch ligand can be aqueous form.

In some embodiments, the kits contain additional components to assist in carrying out the formation of the composition. These include, but are not limited to, photoinitiators, solvents, buffers, plates or other support surfaces, and instructions.

In certain embodiments, the kit further comprises cells to be bound to the composition. In other embodiments, the kit further contains the instruments to be used to harvest autologous cell that are to be bound to the composition. Any of the cells disclosed herein are suitable to use in the disclosed kits.

In some embodiments, the cells can be in a frozen state. The kit further contains a coolant to maintain the cells in a frozen/cryopreserved state. One with skill in the art would understand how to maintain cells in a cryopreserved state. Coolants for use in the present kits include, but are not limited to, ice packs, dry ice, liquid nitrogen or any combination thereof.

When ranges are used herein for physical properties, such as molecular weight, or chemical properties, such as chemical formulae, all combinations, and subcombinations of ranges for specific embodiments therein, are intended to be included.

The disclosures of each patent, patent application, and publication cited or described in this document are hereby incorporated herein by reference, in its entirety.

Those skilled in the art will appreciate that numerous changes and modifications can be made to the preferred embodiments of the disclosed compositions, methods, and kits, and that such changes and modifications can be made without departing from the spirit of the invention. It is, therefore, intended that the appended claims cover all such equivalent variations as fall within the true spirit and scope of the disclosed compositions, methods, and kits.

### EXAMPLES

### Example 1

### Immobilization of Jagged-1 to a Biocompatible Substrate

Diethylene glycol diacrylate and isobutylamine were mixed together at a 1.2:1 molar ratio for 40 hours at 90° C., followed by addition of 0.5 wt % photo-initiator DMPA. The resulting diethylene glycol diacrylate isobutylamine biocompatible substrate was coated onto 24 well plates, and photopolymerized with ultraviolet light (365 nm).

For direct Jagged-1 immobilization, recombinant rat Jagged-1/Fc chimera protein, diluted in PBS, was adsorbed to the diethylene glycol diacrylate isobutylamine surface at 0, 2.5 and 10 μg/ml (Direct[0], [2.5], and [10], respectively) for 2 hours at room temp. The amount of successfully immobilized Jagged-1 and the release kinetics were evaluated via an enzyme-linked immunosorbent assay (ELISA).

For indirect Jagged-1 immobilization, rabbit anti-human Fc antibody was first adsorbed to the diethylene glycol diacrylate isobutylamine surface at 15 μg/ml for 2 hours at room temp, followed by 1% BSA blocking for 2 hours. Jagged-1/Fc was then bound to the anti-Fc antibody at 0, 2.5 and 10 μg/ml (Indirect[0], [2.5], and [10], respectively) for 2 hours at room temp. The amount of successfully immobilized Jagged-1 and the release kinetics were evaluated via an ELISA.

### Example 2

### Effect of Jagged-1 Bound Biocompatible Substrate on Notch Signaling, Cell Viability, Osteogenic Gene Expression, Osteogenic Enzymatic Activity, and Calcified Mineral Deposition

### Method

Human mesenchymal stem cells were plated on Jagged-1 bound diethylene glycol diacrylate isobutylamine (A6) at 5000 cells/cm2 and cultured in standard growth media (SGM: αMEM, 20% FBS, 1× L-Glutamine, and 1× Pen/Strep) for up to 7 days. RNA was harvested at days 1, 3, 5 and 7 for real-time quantitative polymerase chain reaction (qPCR) analysis. Alamar Blue, a cell viability assay, was conducted at days 1, 3, 5 and 7. Alkaline phosphatase, an enzyme produced by osteoblasts during bone formation, was evaluated via histochemical staining at day 7. Human mesenchymal stem cells were also plated on Jagged-1 bound diethylene glycol diacrylate isobutylamine at 10,000 cells/cm2 and cultured in osteogenic media (OGM: αMEM, 10% FBS, 1×1-glutamine, 1× pen/strep, 200 μM ascorbic acid 2-phosphate, 100 mM β-glycerophosphate, 100 nM dexamethasone) for up to 13 days. Alamar Blue was conducted at days 1, 7, 10 and 13. Calcified mineral deposition was evaluated by Alizarin Red staining.

Results

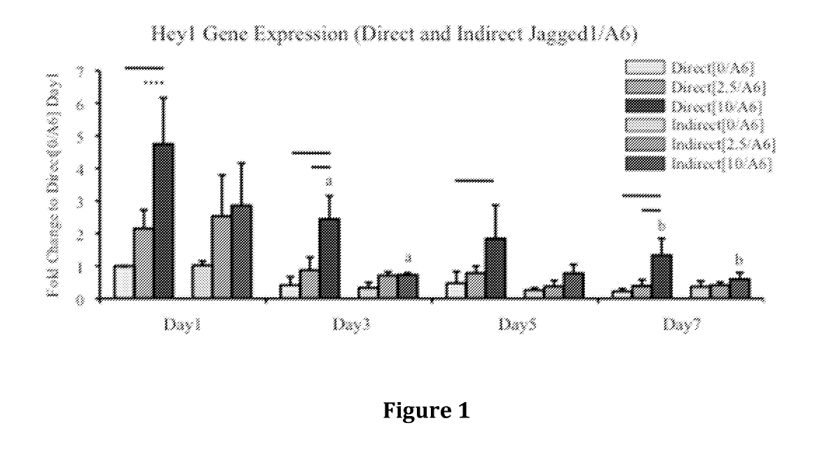

To evaluate the ability of each Jagged-1 immobilization strategy to A6 to activate the Notch signaling pathway, hMSCs were cultured in SGM and harvested at days 1, 3, 5 and 7 for gene expression analysis of Notch target gene Hey1. Overall, Jagged-1 transiently upregulated Hey1 gene expression in a dose-dependent manner (FIG. 1). Specifically, Direct[10/A6] increased expression relative to Direct[0/A6] at all time points and relative to Direct[2.5/A6] at days 1, 3 and 7. Indirect Jagged-1/A6 did not increase Hey1 gene expression for any concentration at any time point. Furthermore, comparing across immobilization strategies, Direct[10/A6] increased expression relative to Indirect[10/A6] at days 3 and 7, demonstrating that the direct immobilization strategy was more effective at activating the Notch signaling pathway. As shown in FIG. 1, Jagged-1 immobilized to the PBAE polymer A6 transiently upregulates expression the Notch target gene Hey1 in hMSCs in a dose dependent response, with direct more effective than indirect.

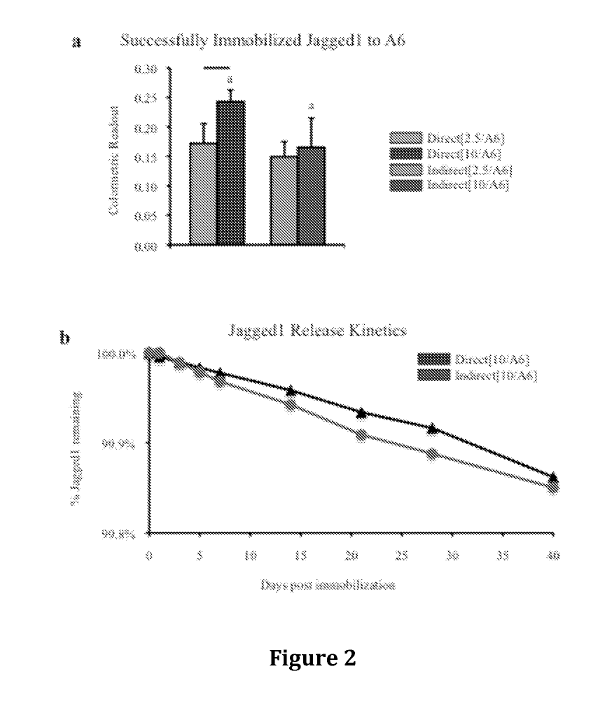

To determine the mechanism responsible for increased Notch activation via the direct method, the relative surface density of successfully immobilized Jagged-1 to A6 was quantified for both strategies. More Jagged-1 was immobilized to A6 via the direct method at 10 μg/mL than the indirect method (FIG. 2A). The direct strategy also increased the amount of Jagged immobilized in a dose-dependent manner, with Direct[10/A6] greater than Direct[2.5/A6]. There was no difference between Indirect[10/A6] and Indirect[2.5/A6]. The release kinetics profile showed that less than 0.2% of successfully immobilized Jagged-1 was released into the incubation media (PBS bath) over 40 days for both direct and indirect methods. (FIG. 2B)

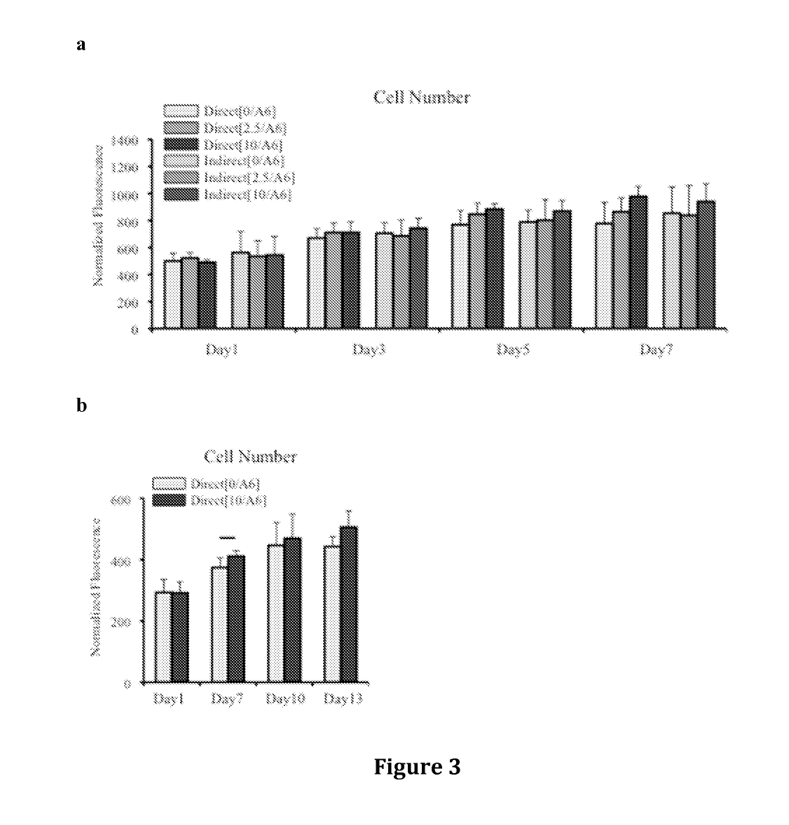

An Alamar Blue assay was used to assess the effects of direct and indirect Jagged-1 immobilization strategies to A6 on hMSC cell number, indicative of proliferation, at days 1, 3, 5, and 7 during SGM culture. Although hMSC number gradually increased over time for all groups, there was no significant effect of Jagged-1 dose or immobilization strategy on cell number at any time point analyzed (FIG. 3A). However, when cultured in OGM, Direct[10/A6] increased cell number relative to Direct[0/A6] at day 7 (FIG. 3B), indicating that Jagged-1 may also promote cell proliferation in an osteogenic environment. As shown in FIG. 3, hMSCs cultured on direct and indirect Jagged-1/A6 constructs in standard growth media or osteogenic media are viable and grow over time.

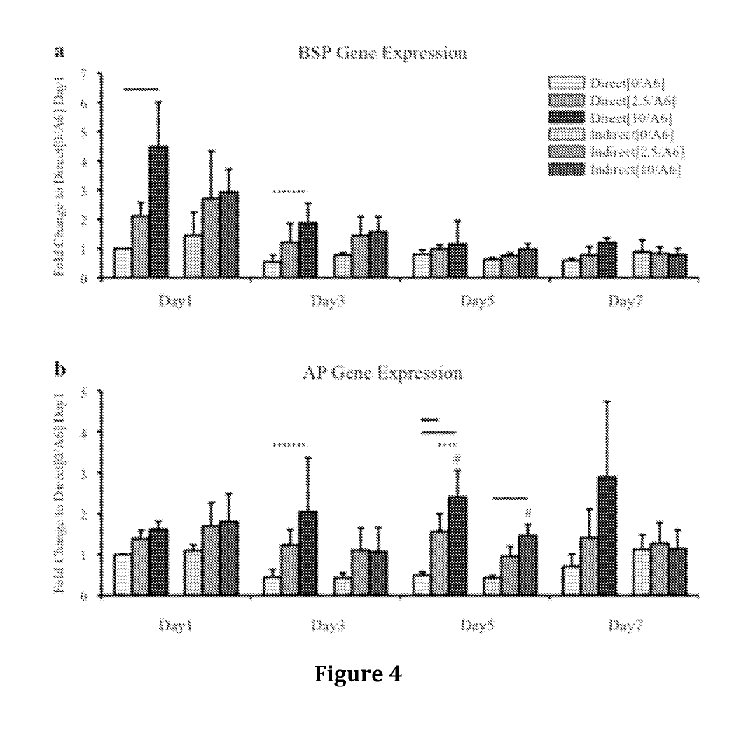

To evaluate the ability of direct and indirect Jagged-1 immobilization strategies to A6 to promote an osteogenic phenotype, bone sialoprotein (BSP) and alkaline phosphatase (AP) gene expression were quantified at days 1, 3, 5 and 7 during SGM culture. Overall, Jagged-1/A6 increased BSP gene expression in a dose-dependent response (FIG. 4A). Specifically, Direct[10/A6] increased BSP expression relative to Direct[0/A6] at days 1 and 3. Indirect Jagged-1/A6 also did not increase BSP gene expression for any concentration at any time point. Overall, Jagged-1/A6 also increased AP gene expression, with Direct[10/A6] increased relative to Direct[0/A6] at days 3 and 5, and relative to Direct[2.5/A6] at day 5 (FIG. 4B). Direct[2.5/A6] was also increased relative to Direct[0/A6] at day 5. Indirect[10/A6] increased AP gene expression relative to Indirect[0/A6] at day 5. However, comparing across immobilization strategies, Direct[10/A6] was increased relative to Indirect[10/A6] at day 5. Collectively, the data demonstrates that the direct immobilization strategy was more effective at inducing osteogenic gene expression.

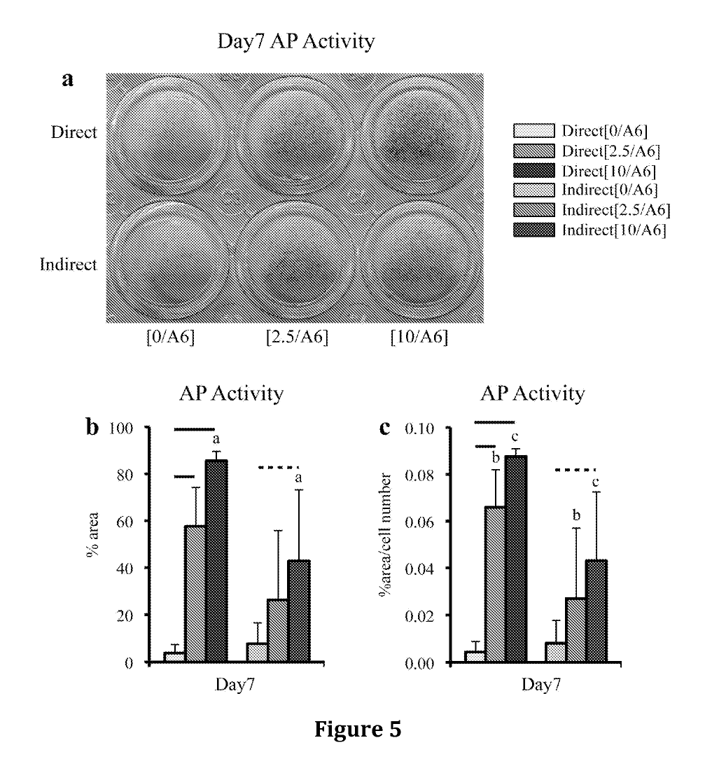

hMSCs were also stained for AP enzymatic activity at day 7 during SGM culture (FIG. 5A). Overall, Jagged-1/A6 increased AP activity in a dose-dependent manner (FIG. 5B). Direct[10/A6] and Direct[2.5/A6] were increased relative to Direct[0/A6]. Indirect[10/A6] was also increased relative to Indirect[0/A6]. However, comparing across immobilization strategies, Direct[10/A6] increased AP enzymatic activity relative to Indirect[10/A6], demonstrating that the direct immobilization strategy was more effective at inducing osteogenic enzymatic activity. Similar results were also found for AP staining normalized to cell number, demonstrating increased osteogenic activity for direct Jagged-1/A6 on a per cell basis (FIG. 5C). Thus, Jagged-1 increases AP enzymatic activity, specifically % area of staining (FIG. 5B) and % area of staining normalized by cell number (FIG. 5C), with direct more effective than indirect.

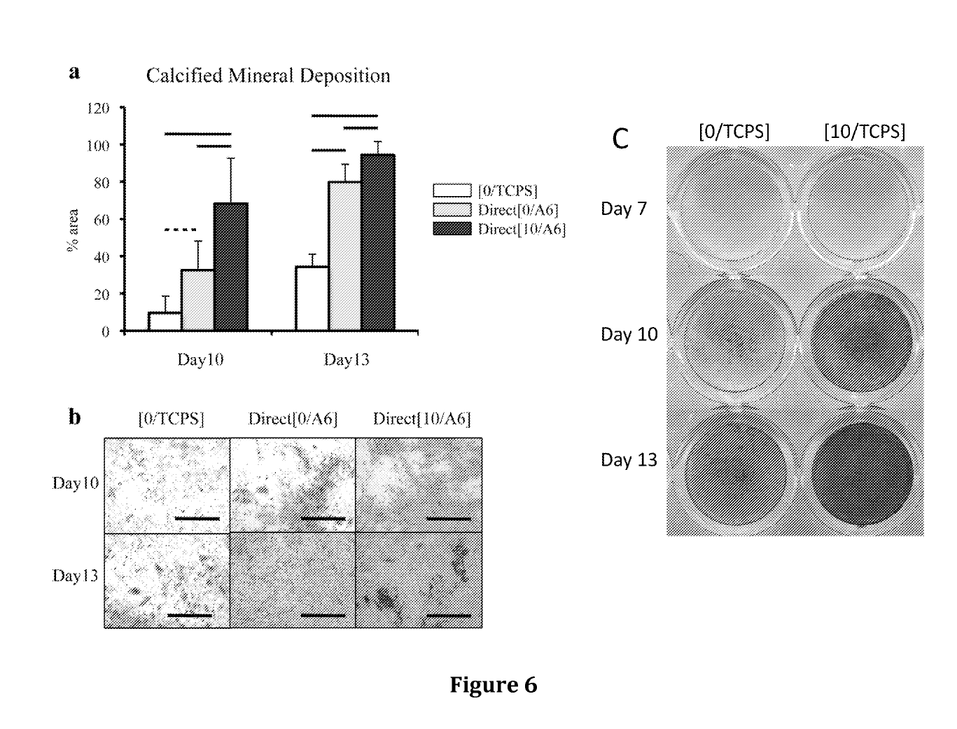

Alizarin Red staining of calcified mineral tissue deposition by cells was conducted at days 10 and 13 to evaluate the ability of direct Jagged-1 immobilization to A6 to induce terminal osteoblast differentiation in hMSCs. Direct[10/A6] increased calcified mineral deposition relative to Direct[0/A6] at all time points (FIG. 6A-B). Collectively, the data demonstrates that direct Jagged-1/A6 induces osteoblast differentiation and calcified mineral deposition. The experiment was also repeated for Jagged-1 directly immobilized to another substrate, tissue culture plastic (TCPS). Similarly, Jagged-1 immobilization increased the amount of calcified mineral deposition at days 10 and 13 (FIG. 6C), demonstrating that Jagged-1 immobilized to several substrates induces osteoblast differentiation.

### Example 3

### Administration of Notch Ligand Bound Biocompatible Substrates

The Notch ligand bound biocompatible substrate can be formulated under conditions suitable for administration into a patient. If implantation of the composition is required, the composition can be formed using a biocompatible substrate comprising a solid support. If injection is required, the composition can be formed using a biocompatible substrate comprising an aqueous solution, gel, hydrogel, bead, or nanoparticle.

Following formation of the Notch ligand bound biocompatible substrate, cells can be added and allowed to adhere to the composition for approximately 30 min to 1 hour.

For implantations, the composition can be formulated as a solid support, the wound or surgical site can be opened, and the composition can be implanted into the defect space. If necessary, the implant can be secured in place using appropriate procedures. The wound can then be sutured closed.

For injections, such as in a closed wound, the composition can be formulated in an injectable form and injected into or proximate to the defect space. The injection site can be determined by estimation of the surgeon, or through image guidance of an X-ray, CT, MRI, ultrasound, or other imaging modality.

### Example 4

### Wild Type Mice with Endogenous Jagged-1 Expression have Increased Trabecular Bone Formation

### Method

Jag1^{f/f} mice were crossed with mice expressing Cre recombinase on the Prx1 promoter (Prx1-Cre+;Jag1^{f/f}). In this model, Jag1 is conditionally deleted in mesenchymal progenitor cells of the limb-bud prior to skeletal development. Wild type mice are heterozygous and homozygous Jag1 floxed but Cre-negative (WT). These mice are on a C57Bl/6 background. Jag1^{f/f} mice were also crossed with mice expressing Cre recombinase from the 2.3 kb fragment of the collagen type I promoter, also known as the Col2.3 promoter (Col2.3-Cre+;Jag1^{f/f}). In this model, Jag1 is deleted in an osteoblast-specific population later on during differentiation. Wild type mice are heterozygous and homozygous Jag1 floxed but Cre-negative (WT). These mice are on a mixed C57Bl/6 and CD1 background.

### Results

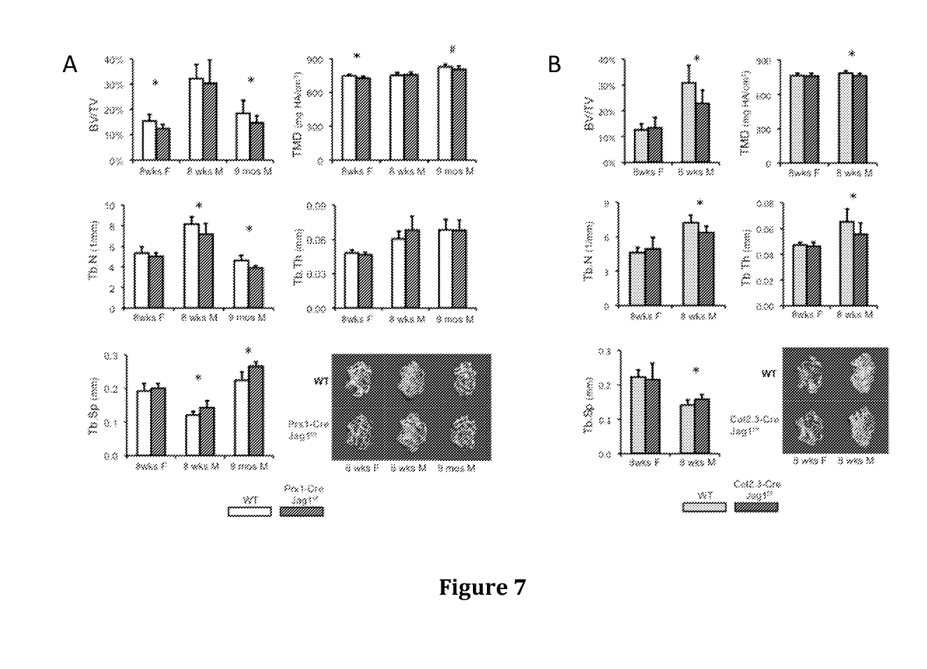

Femurs were harvested for micro-computed tomography analysis of bone formation from Prx1-Cre;Jag1^{f/f} and Col2.3-Cre;Jag1^{f/f} male and female mice with respective WT controls at 8 weeks of age. Additional femurs from Prx1-Cre;Jag1^{f/f} male mice and WT mice were also harvested at 9 months (n=7-12). As shown in FIG. 7, wild type (WT) mice with endogenous Jagged-1 (Jag1) expression have increased trabecular bone formation, measured by micro-computed tomography, relative to mice with Jagged-1 conditionally deleted in mesenchymal progenitor cells (A: Prx1-Cre Jag1^{f/f}) or committed osteoblasts (B: Col2.3-Cre Jag1^{f/f}).

### Example 5

### Jagged-1 Activity is Positively Correlated with Osteoblast Activity

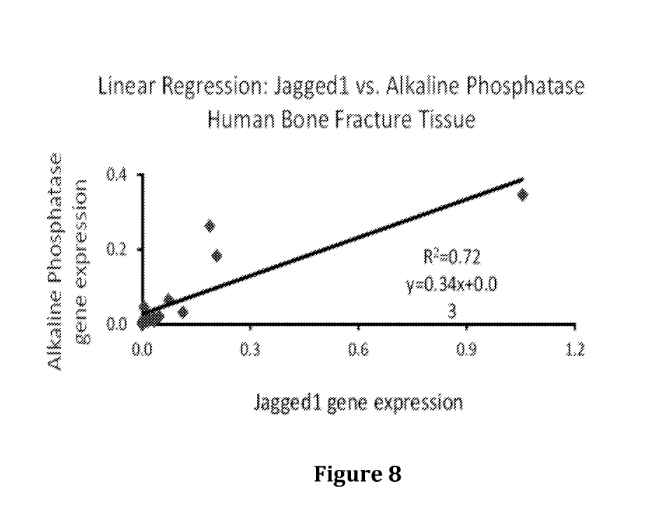

Human bone fracture tissue (shredded bone and marrow) was extracted at the time of surgical fixation in the operating room. RNA was extracted from each sample and underwent gene expression analysis via real time quantitative polymerase chain reaction. As shown in FIG. 8, Jagged-1 activity is positively correlated with osteoblast activity (Alkaline Phosphatase gene expression) during human bone fracture repair.

### Example 6

### Delivery of Jagged-1 with an Osteoconductive Scaffold Stimulates In Vivo Human Mesenchymal Stem Cell Mediated Bone Formation

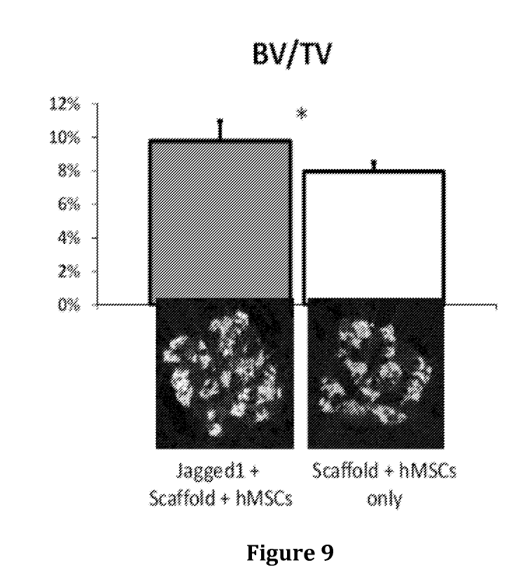

33 ug of recombinant rat Jagged-1 diluted in PBS was added to a 3×3×3 mm³ Collagraft® scaffold (Zimmer)—comprised of hydroxyapatite, beta-tricalcium phosphate, and collagen—and allowed to adsorb for 2 hours. Control constructs received PBS only. 10⁶ bone marrow-derived human mesenchymal stem cells (hMSC) were then loaded onto the scaffold. Jagged-1-scaffold-hMSC and control-scaffold-hMSC constructs were then implanted subcutaneously under the dorsum of immunocompromised (SCID) mice (n=7). Constructs were harvested 8 weeks later. Bone formation was evaluated via micro-computed tomography. As shown in FIG. 9, delivery of Jagged-1 with an osteoconductive scaffold stimulates in vivo human mesenchymal stem cell (hMSC) mediated bone formation, measured by micro-computed tomography, relative to the scaffold with hMSCs only.

### Example 7

### Jagged-1 Bound Biomaterial Increases Bone Regeneration

### Method

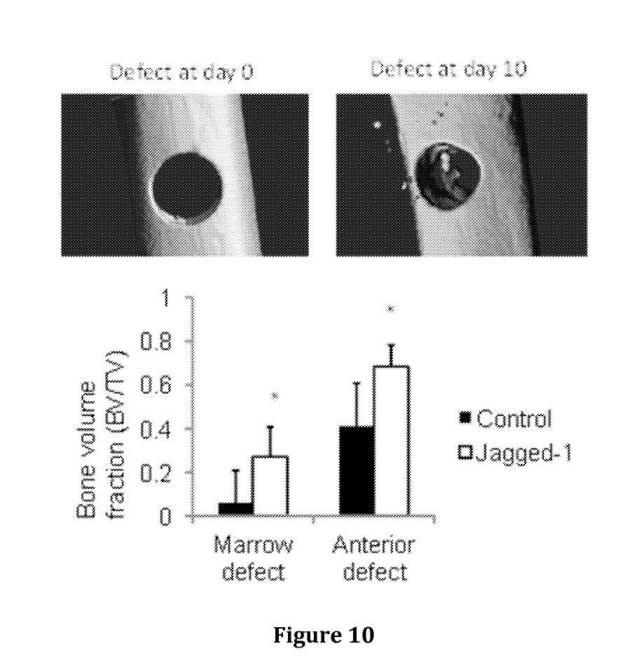

Rat Jagged-1 (6 μg in PBS—15 microliters of 400 μg/mL of recombinant rat Jagged-1 Fc fusion) was bound to a resorbable collagen matrix that was cut into a 1 mm diameter cylinder. PBS was used as a control. 0.8 mm femoral cortical bone defects were created in both femurs of ten B6 mice and then one femur was treated with Jagged-1 and the other was treated with control by tight-fitting the collagen matrix into the anterior hole and into the marrow cavity. Five mice were harvested at 10 days post-treatment and five mice were harvested at 20 days post-treatment. Both sets of bones were analyzed using microCT and the amount of new bone determined (FIG. 10).

### Results

Defects treated with Jagged-1 had much greater new bone formation in both the anterior cortical hole (anterior defect) and in the marrow cavity (marrow defect).

### Example 8

### Surface-Bound Jagged-1 Directly Induces hMSC Osteoblast Differentiation

### Methods

Primary hMSC were isolated from marrow and cultured in assay medium with 20% defined FBS (Hyclone, Logan, Utah, USA). Assay medium is supplemented McCoy's 5a medium (Invitrogen). Osteoblast differentiation media was serum-free plus 25 mg/ml ascorbic acid and 5 mM beta-glycerol phosphate. Following treatments and osteoblast differentiation, cells were harvested for Alkaline Phosphatase enzymatic histochemical activity and Alizarin Red S staining. As well, RNA was harvested for gene expression profiling using quantitative PCR (not shown).

Immobilized Jagged-1 was used in treatment of MSC as described in Zhu F, Sweetwyne M T, Hankenson K D. 2013. Stem Cells 31: 1181-92. Briefly, tissue culture plates were pre-coated with 10 ug/mL of antibody against the Fc portion of human IgG (Jackson ImmonoResearch) for 1 hour. Excess antibody was removed and then wells were incubated with the indicated concentration of recombinant rat Jagged-1/human Fc IgG chimeric protein for 2 hours. Control plates were incubated with human Fc IgG only or untreated as indicated. MSCs were then plated to the pre-coated plates.

### Results

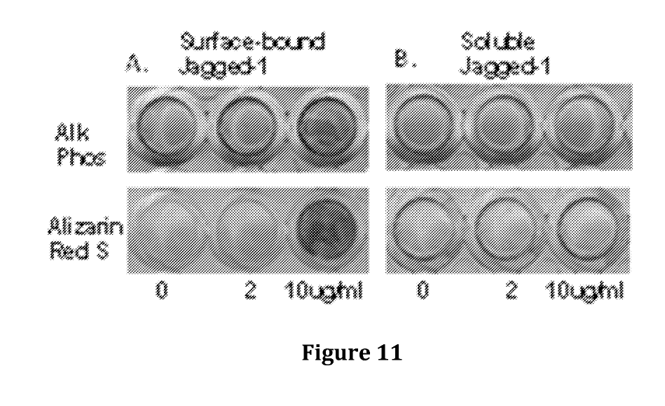

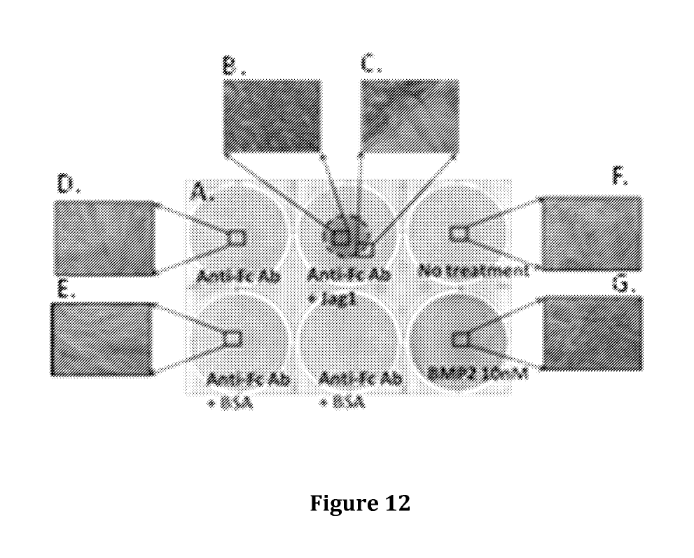

As shown in FIG. 11, plate-bound Jagged-1 (FIG. 11A)—but not soluble Jagged-1 (FIG. 11B)—was able to induce osteoblast differentiation. The expression profile of osteogenic (ALP, Osterix, BSP, DMP) and Notch target genes (HEY/HES) are consistent with the cell-staining results (not shown). Only hMSC that were in direct contact with Jagged-1 were able to develop as osteoblasts (FIGS. 12B and 12C).

### Example 9

### Local Activation of Notch Signaling Enhances Geriatric Bone Regeneration

### Methods

Bilateral tibial fractures were induced in 21 young adult (5 mo old) and 21 geriatric (25 mo old) C57BL/6 mice, followed by an injection with NICD-eGFP (Notch Intracellular Domain-enhanced Green Fluorescent Protein)-adenovirus on 5 DPF (Days Past Fracture). After 10, 20 or 40 DPF these tibiae were harvested and analyzed using MicroCT (μCT) and histology. qPCR was used to verify viral construct expression. Unpaired, 1-sided T-tests were used to compare results from the young and geriatric mice.

For this experiment male C57BL/6 mice (“black six”) were used, which were ordered from the National Institute of Aging (NIA). A total of 42 mice were used, divided into 2 age groups: Young adult mice (aged 5 months) and senescent mice (aged 25 months). These 2 groups were further divided into 3 subgroups (n=7 per group): a group of which the tibiae were harvested on 10 DPF (days past fracture), on 20 DPF or on 40 DPF. All mice were properly taken care of, according to IACUC (Institutional Animal Care and Use Committee) protocols.

All mice were operated in a sterilized fume hood, anesthetized using isoflurane, given an injection of buprenorphine and saline to counter any blood loss, and provided an analgesia. With their knees in 90 degrees flexion, a small incision was made medial to the tibial tuberosity, after which a 26 gauge needle was gently inserted in the cortex at a 45 degree angle to the tibia by slightly twitching the needle. Upon entering the medulla, the needle was then brought parallel to the tibia and with minimal force driven distally. The needle was taken out and replaced with a 0.009 inch intramedullary pin. After a thorough check to see if neither the needle nor the pin punctured the bone or the skin, the pin was cut to size and the skin was glued back together using Tissumend II adhesive glue (Veterinary Products Laboratories), again making sure that the pin did not stick out. Whilst still being anaesthetized, the mice were then placed on a custom-made three point bending apparatus to fracture the tibia mid-diaphysis. With this machine it is possible to give roughly the same force on each bone, thereby making the fracture procedure more standardized. Post-surgically, the mice were given two-daily doses of buprenorphine for 3 days.

Five days post fracture (DPF) the right legs of each mouse was injected with 30 μl of NICD-eGFP virus (suspended in PBS) and the left leg was injected with 30 μl of GFP virus (suspended in PBS). Both injections contained 1×10⁹ viral particles. Prior to injection, the mice were anaesthetized and prepared as if they were undergoing surgery, meaning they were placed on sterile underpads and received a continuous inflow of isoflurane.

The murine tibiae were harvested at 10, 20 and 40 DPF. Mice were euthanized in a carbon dioxide room followed by manual cervical dislocation, as per IACUC protocol. Right after death, cardiac puncture took place and blood was collected for possible future use. Approximately 10 ml of blood could be extracted from a single sample. The tibial-fibular complex was extracted, making sure that there was as little as possible muscle and ligaments on the bone, without compromising the callus. After extraction, the tibial-fibular complex was put on 4% PFA. After 3 days the PFA was replaced by 70% ethanol.

Real-Time PCR was performed using the 7500 Fast Real-Time PCR System (Applied Biosystems), to evaluate expression of Notch1 and GFP, using β-actin as a housekeeping gene. The Notch1 primer was used to check whether or not NICD was upregulated, since the Notch1 primer binds to the region that encodes for NICD.

MicroCT (μCT) analysis was performed using the VivaCT 35 μCT scanner by SCANCO Medical AG. The harvested bones were batch scanned; by placing the bones in a custom sized tube, up to 4 bones could be scanned at a time. 10 DPF bones were wrapped in dry gauze, whereas 20 DPF and 40 bones were wrapped in ethanol soaked gauze. The following parameters were used: FOV (Field of view)/diameter (in mm)—21.5, voxel size (in nm): 21, thickness of slices: 0.02 mm, number of projections per 180°: 125. The analysis of the scanned bones was performed on the same computer as the actual scans. Total callus volume, callus bone volume, bone volume fraction (callus bone volume divided by total callus volume), tissue mineral density, trabecular number, trabecular thickness, trabecular separation, connectivity density and structure model index were analyzed. In order to obtain these parameters, scans were first processed. Upon loading the scans, the callus starts and ends were established. The callus was manually outlined every tenth slide. The morphing ability of the analysis program was used to automatically draw the lines in the ‘in between’ slides. If the size of the callus changed dramatically between 10 slides, smaller steps were used. Upon drawing outlines in all slides, the outlines were saved as a .GOBJ file and the steps were repeated, outlining the bone and marrow to exclude actual bone (since only calcium in the callus was of interest). Once all the bone was excluded, the ‘inclusion’ and ‘exclusion’ drawings were combined and saved. The program automatically analyzed these pictures. For the 10 DPF bones a square around the (expected) callus was drawn, since there was not yet macroscopic calcification in the callus at that time.

Following μCT, the pathology of the samples were evaluated. The bones were decalcified and sliced for histology, making sure that every slide showed both the callus at its thickest point and the knee-joint of the proximal tibia, as well as the distal part of the tibia. Samples were stained with Safranin O, Fast Green and Hematoxylin, which stain red for keratin and cartilage and green for background color. Since the joint contained cartilage as well, this could be used to verify that the staining worked. Decalcification by pathology was established by submerging the tibiae in 15% formic acid, followed by flushing with water. For the staining the slides were first cleared (by submerging the slides in 2×5 minutes of Histoclear©), followed by a gradual dehydration of the bones using ethanol (2×2 minutes of 100% ethanol, followed by 2×2 minutes of 95% ethanol and 1×2 minutes of 75% ethanol). After these steps the slides were dipped in ddH₂O (double distilled water), after which they were bathed in Hematoxylin for 5 minutes (used to stain nuclei), followed by another ddH₂O dip bath. The slides were submerged for 3 minute in 0.3% Fast Green and 0.5% acetic acid (the former stains the histones, whereas the latter is used to extract acidic DNA), followed by another 2 minutes of 1% acetic acid. The slides were stained with 5.45% Safranin-O for 30 seconds (Saf-O stains cartilage), followed by dip baths and submerging in ddH₂O (2 minutes) for flushing. The slides were gradually dehydrated again with ethanol (1×2 minutes of 75% ethanol, followed by 2×2 minutes of 95% ethanol and 2×2 minutes of 100% ethanol). Finally, the slides were submerged in Histoclear (2×5 minutes) to clear the slides and to prepare them for permounting and a cover slip. The cover slip was secured. Analysis was performed using an Olympus BX51 light microscope attached to a SPOT RT3 two mega-pixel camera attached to a computer, which enabled photos to be taken. Photos were taken of the entire length of the bone at a 2× magnification, using the following parameters: Exposure times: 1.4 (Red), 1.744 (Green) and 2.176 (Blue) and 0.75 (Gamma) These photos were saved as a .tiff file and were stitched together using Microsoft Image Composite Editor. The now stitched photos were loaded into ImageJ (National Institutes of Health, Bethesda, Md.). With this program the outline of the callus was drawn, measuring the size of the callus. Using a color thresholding tool, the amount and percentage of cartilage tissue was objectified.

Data from the μCT was compiled into 3 Excel© sheets, one for each DPF point. Means and standard deviations were calculated using Excel, followed by unpaired, one tailed student's T-tests, comparing GFP and NICD in young mice and in old mice. One tailed T-tests were chosen for μCT analysis. Two tailed T-tests for the cartilage parameters. After these initial statistics, data was manually transferred to IBM's SPSS 14© for making the graphs. Data from histology was compiled into 3 Excel sheets, and means and standard deviations, as well as unpaired, one tailed student's T-tests, were calculated. Again, the data was then transferred manually to SPSS14 for proper graphing. PCR data was imported to Excel. Each gene of interest was measured triplicate, allowing the raw ddCT (Delta-Delta Cycle Threshold) data and averages to be calculated in every sample. The average β-Actin values were then used to standardize the average GFP and average NICD values, using the formula: 2^{-ΔC(t)}, where C(t) is β-Actin minus GFP or β-Actin minus NICD. Averages and unpaired, two-tailed T-tests (comparing NICD to the uninjected leg or GFP to the uninjected leg) were calculated using Excel.

### Results

GFP showed significance in the GFP-injected leg (p=0.0003) where it was expressed 14.39 times higher than in the uninjected leg. GFP expression was also significantly higher in the NICD-injected leg (p=0.017) where it was expressed 221.14 times higher than in the uninjected leg. GFP-injected legs did not show any significant expression of NICD (p=0.338) with their uninjected controls. NICD did however show a significant difference (p=0.022) in the NICD-injected legs, with a 71.58 times increased expression compared to the uninjected control leg.

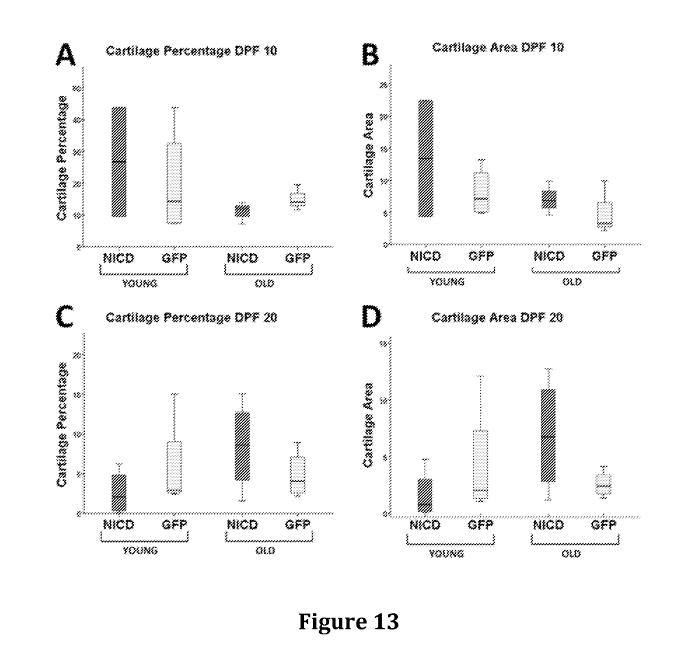

Using Safranin-O and Fast green stained histology slides, the size of the cartilaginous callus and the percentage of cartilage in the callus were determined. While no significance was found, a trend towards significance was observed. At 10 DPF, the young NICD legs had on average a 65.31% larger cartilaginous area with a 34% increase in cartilage percentage compared to their GFP counterparts. (Table 1 and FIGS. 13A and 13B) In the old 10 DPF mice, a difference in favor of NICD over GFP was observed. The average cartilage area in NICD legs was 40% larger. The cartilage percentage in the NICD mice is lower than the GFP mice however. Cartilage percentage is calculated by looking at total callus area and calculating the percentage of cartilage area in that callus. Since the mean total callus area in the NICD mice was so large (86.50% larger than the GFP mice), the difference between total callus area and cartilage area in NICD mice was larger than the difference between total callus area and cartilage area in GFP mice, resulting in a net smaller cartilage percentage in the NICD mice. (Table 1 and FIGS. 13A and 13B) Interestingly, at 20 DPF the young NICD mice have a 170% smaller cartilage area with a 125% smaller cartilage percentage than the GFP control group. (Table 1 and FIGS. 13C and 13D) Contrary to the young 20 DPF mice, the old (20 DPF) NICD mice still show a larger cartilage area (160% larger than GFP mice) and a larger cartilage percentage in that callus (76.36% larger than GFP mice). (Table 1 and FIGS. 13C and 13D) As expected, 40 DPF mice (regardless of age and virus) did not show cartilage in their calluses. That these calluses were in fact cartilage free (and not just a staining error) was confirmed by looking at the joints of these bones. The cartilage at the joints did stain, thereby verifying that indeed the 40 DPF mice lost the cartilage in their calluses because the calluses have been calcified.

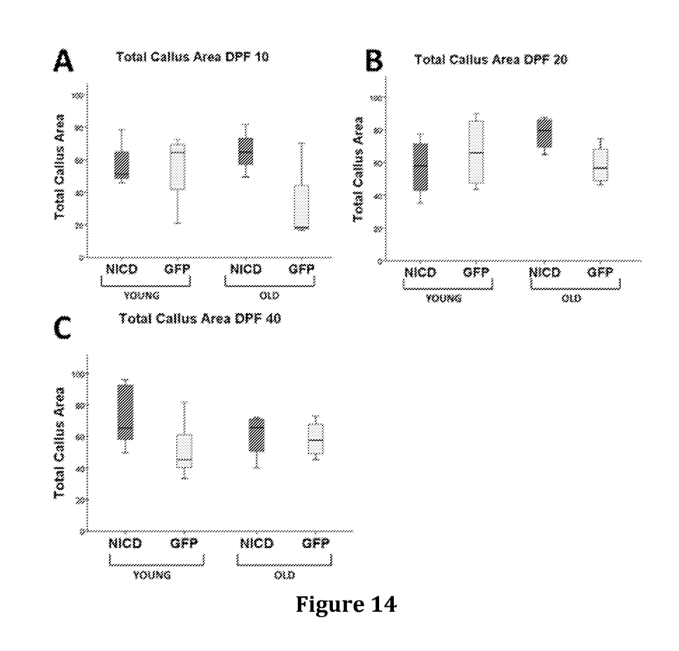

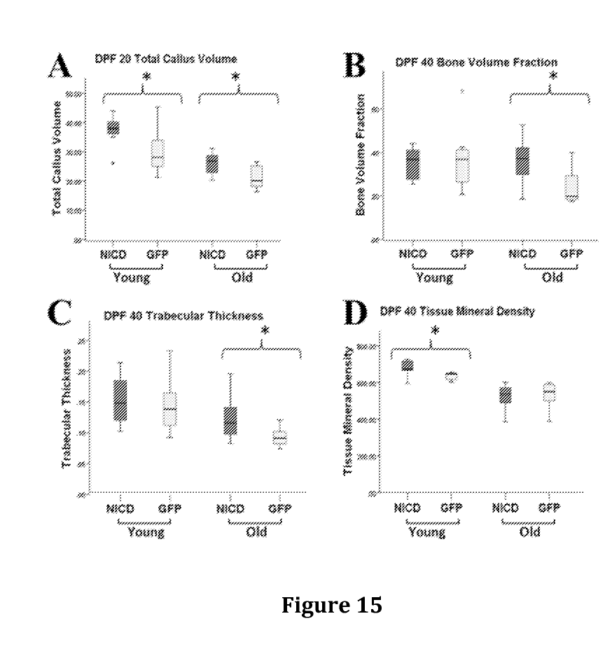

Callus size was measured both using histology and μCT. μCT measures and shows calcification. Since 10 DPF mice do not yet have a macroscopically visible callus, the callus could not simply be outlined to calculate the amount of bone within. To overcome this problem, a square was used to outline the bone and callus. Because of this, no μCT data regarding total callus size was available. The callus area, however, was measured using the histology samples. Callus size is measured in mm³ (μCT) or mm² (histology). In the young 10 DPF group there was a 5.35% larger callus seen in the NICD group. In the old 10 DPF mice the differences were even bigger, with a 86.50% larger callus in the NICD group compared to the GFP group. (Table 1 and FIG. 14A) A significantly larger callus was observed in both the young and old 20 DPF NICD groups. μCT shows both young and old NICD mice have a 23% larger callus (young: p=0.049, old: p=0.028). (Table 2 and FIG. 15A) Surprisingly, the young 20 DPF NICD mice showed a (non-significant) 16% smaller callus on histology. The old 20 DPF NICD mice had a 32.85% larger callus on histology. (Table 1 and FIG. 14B) The μCT results of the 40 DPF mice showed that the young mice have a small (non-significant) increase in total callus volume of 14%. The old 40 DPF NICD mice show a slightly smaller callus than their GFP control group (3.81% smaller). (Table 2) NICD callus area was larger in 40 DPF histology samples as well, with a 38.30% larger callus area in the young group and a slightly larger callus area of 4.06% in the old 40 DPF group. (Table 1 and FIG. 14C)

The amount of calcium can be quantified using a number of parameters. Using μCT the bone volume (in mm³), tissue mineral density (amount of hydroxy apatite per cm³) and bone volume fraction (fraction of bone volume in total callus volume) was measured. Since the callus area in the 10 DPF mice could not be outlined, no bone volume fractions were available for this time point. Young 10 DPF mice did not show clear differences regarding calcification parameters between NICD and GFP. Old 10 DPF mice did, however, show large (though non-significant) differences, with a 53% higher bone volume in NICD calluses and a 36% higher tissue mineral density. (Table 2) Comparing NICD and GFP calluses in young 20 DPF mice, very small non-significant differences in bone volume fraction and tissue mineral density were observed. The NICD mice did show 25% larger bone volumes than the GFP control group. The old 20 DPF group showed small, non-significant, differences between NICD and GFP, most notably a 19% larger bone volume in the NICD group. (Table 2) Turning our attention to 40 DPF mice, a significant increase in tissue mineral density (p=0.019) of 7% in young NICD calluses was observed. Non-significantly larger bone volumes (11%) were also seen in the young NICD mice. (Table 2 and FIG. 15D) The old 40 DPF NICD mice showed significantly (p=0.030) higher bone volume fractions (39% higher) and non-significant increases in bone volume (36% higher). (Table 2 and FIG. 15)

Apart from measuring the size of the callus and the amount of calcified tissue in said callus, μCT can be used to provide information about bone morphometry, meaning it can tell us something about how the bone (callus) is built up and what it consists of μCT measures trabecular number (amount of trabeculae per mm³), trabecular thickness (average thickness of trabeculae, measured in mm³) and trabecular separation (size of the gaps between trabeculae, measured in mm³) It also measures connectivity density (amount of trabecular connections per mm³) and finally SMI or Structure Model Index. SMI is a number between 0 and 3 and gives us information about the shape of the trabeculae. A 0 means that all the trabeculae are horizontally plated, and a 3 means that all the bones are aligned in cylindrical rods. Rods are typically seen in the elderly, in more brittle bone (such as in osteoporosis) and in fractures. Rods bind to multiple trabeculae but have limited marrow binding, whereas plates have multiple attachment sites for marrow, with fewer sites for other trabeculae.

At 10 DPF, young NICD mice showed a higher trabecular number (14% higher) than the GFP mice. NICD also has a 43% higher connectivity density than its control group. In the old 10 DPF mice, NICD had a 50% higher trabecular thickness than GFP. (Table 2) At 20 DPF, young mice did not show notable differences in morphometrics, the old 20 DPF NICD mice however showed a 21% increase in trabecular separation. (Table 2) As with 20 DPF, the 40 DPF young mice too showed little notable differences in morphometry, the NICD group did however had a much higher SMI (1.495 in NICD opposed to 1.020 in GFP). The old 40 DPF mice, finally, showed a significant increase in trabecular thickness (p=0.038) of 29%. (Table 2 and FIG. 15C)

In this study, the fracture healing process was followed in young and old mice, looking at the effects of NICD in said process. Since injections were given on 5 DPF and the first tibial harvests took place on 10 DPF, no data was collected about the inflammatory phase. Like the inflammatory phase, the late remodeling phase of the healing process was outside the study frame since the final time point was 40 DPF. At this time, the callus is entering the remodeling phase, but is not yet far in this phase. Normally the callus would slowly resorb until it has the thickness (and strength) of the rest of the bone. Upregulation of NICD would have meant overstimulation of osteoblast cell lines but at the same time downregulation of osteoclast formation. This would likely mean that the callus stays larger than the rest of the bone, since bone resorption is impaired.

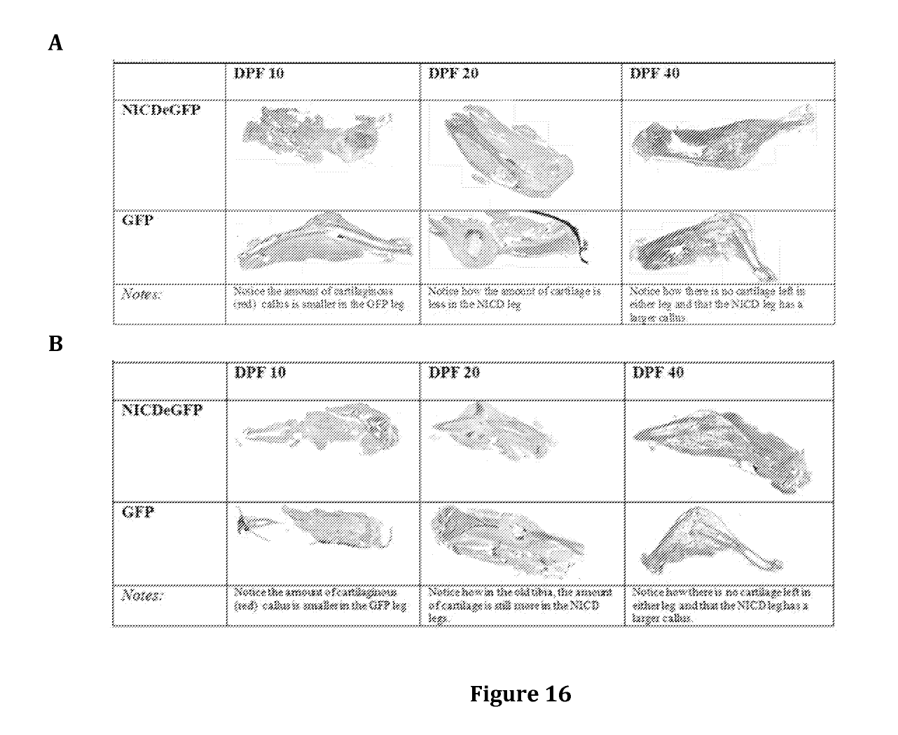

During the inflammatory phase, fibroblasts will make a scaffold of collagen. Chondroblasts will use this scaffold to build what is called a soft callus; a cartilaginous callus. It was hypothesized that NICD will increase healing both in time and in quality. This holds to be true for cartilage formation. Histology showed that young mice have no problem making a cartilaginous callus and that NICD helps them make more cartilage. In healthy young mice (with adequate healing response) the effects of NICD in a larger cartilage area (meaning more proliferation of progenitor cells for chondrogenesis) at 10 DPF are observed. (FIG. 16A; DPF 10) Hypertrophic chondrocytes are needed for calcification of the callus by releasing hydroxyapatite. Our young 20 DPF NICD calluses show far less cartilage at this point (contrary to their GFP controls), because these calluses are already calcified. (FIG. 16A; DPF 20) This means that NICD increases the speed of fracture healing at least up to this point. The young NICD mice have a larger cartilaginous callus early on and move to calcifying the callus faster.

Geriatrics have impaired fracture healing. Processes are still working but at a slower rate. In this study, more cartilage in the old mice at 10 and 20 DPF was observed, (FIG. 16B; DPF 10 and 20), thus NICD boosts cartilage formation, like it did in the young 10 DPF mice. Contrary to the young 20 DPF mice though, the old mice did still have cartilage, in a higher amount than the GFP control group. This means that in old mice NICD has strong proliferation boosting properties (more cartilage), but does not really shorten the cartilaginous phase. Another explanation for the cartilage-rich old DPF 20 NICD calluses is that the beneficial effects of NICD and the detrimental effects of old age collide, giving positive spatial effects (NICD), but not (so much) the temporal effects (old age). In late phases of fracture healing (40 DPF) finally, none of the groups showed any cartilage at callus site. The bones did however stain for cartilage at the joint, confirming that there was no error in staining (FIGS. 16A and 16B; DPF 40).

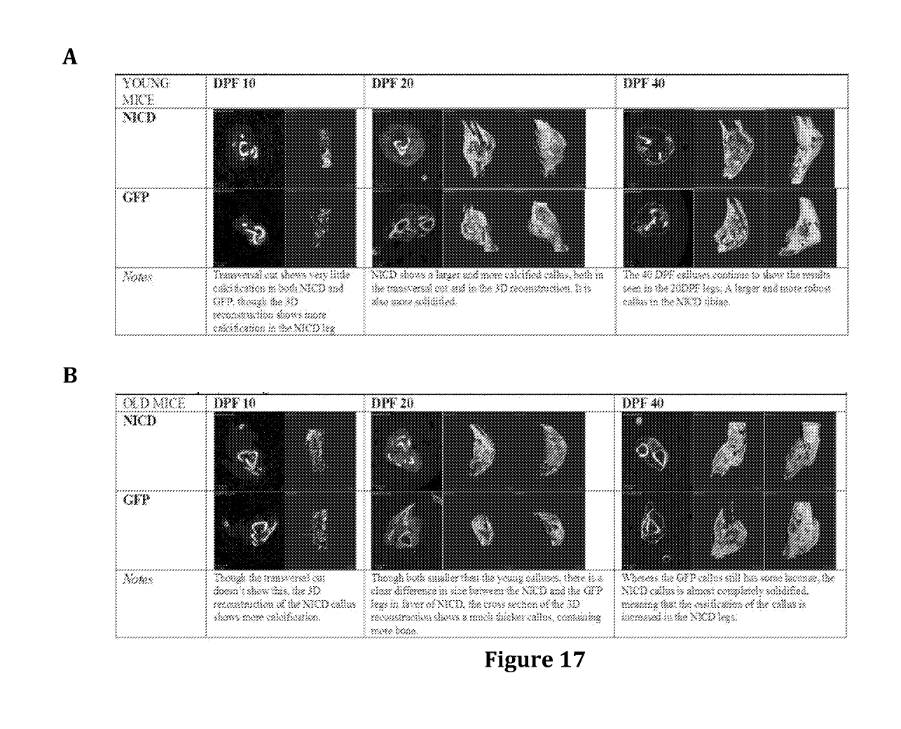

Following cartilage formation, chondrocytes become hypertrophic and start depositing hydroxyapatite. Thus begins formation of the hard callus. Considering that there is overlap between the phases, bone formation starts when there is still cartilage tissue in the callus. In that study, higher trabecular numbers were found in young mice starting at 15-20 DPF. This study shows tendencies towards even earlier trabecular number increases. Looking at young 10 DPF NICD mice, a higher trabecular number and connectivity density, meaning that NICD increases the speed with which the fracture heals, was observed. A higher trabecular number and connectivity density also mean that the callus has more strength. Trabecular number is a good indicator for the strength of the bone. On top of that, young NICD tibia also showed a higher bone volume. (FIG. 17A; DPF 10) Focusing on the geriatric 10 DPF mice, the NICD group has an increased bone volume with increased tissue mineral density. (FIG. 17B; DPF 10) This means that the geriatric group is already starting with hydroxyapatite depositions, chondrocytes are (at least partially) hypertrophic, and that hard callus formation has already commenced. This is interesting because, at 20 DPF, histology slides still showed cartilage, even more than the GFP control group. This suggests that NICD triggered chondrocytes to become hypertrophic (and produce hydroxyapatite) early on, while at the same time still having the slower healing process that is associated with old age. Combining these results with the fact that geriatric mice also showed a higher trabecular thickness under the influence of NICD, it can be concluded that there is an earlier hard callus formation. NICD apparently does speed up calcification in geriatric mice, making the overlap of soft callus and hard callus phase broader. Since the thickness of the trabeculae is increased, it also means that NICD makes the callus stronger, which especially in geriatric fractures is very important. A stronger callus implies that load can be put on it earlier, making rehabilitation faster and muscle atrophy less.

Though tendencies were found in callus size, calcification parameters and cartilage amounts at 10 DPF, the first significant data encountered was at 20 DPF. NICD significantly increased total callus volume, both in young and old mice. (FIGS. 17A and 17B; DPF 20) A larger callus means that it grows faster which implies higher cellular activity. With Notch upregulating the osteoprogenitor cell proliferation, it can be explained that the 20 DPF mice (both young and old) showed increased bone volume as well. Especially in geriatrics this is important, since elderly have a natural decline of callus size and bone volume as they grow older. NICD seems to partially counteract this decline. Trabecular separation is also higher in the old DPF 20 NICD mice. This illustrates that the callus size expands at a faster rate than the trabeculae. This further supports the proliferation boosting capabilities of Notch.

At DPF 40 it was expected the callus to be at a stage where it is solidified sufficiently to bear everyday stress. The only process it now faces is remodeling, which is something that happens over months (years in humans). The callus is still very much in a moving process though, finishing the final steps of making a solid callus and slowly starting the first steps of remodeling. Young DPF 40 NICD mice showed a significantly higher tissue mineral density and an increased callus and bone volume, all pointing towards a more potent healing process. (FIG. 17A; DPF 40) Interestingly, the structure model index (SMI) was (significantly) higher as well. A robust and young bone typically exists of more plate-like trabeculae than rod-like trabeculae and rod-like trabeculae are typically seen in geriatric and osteoporotic bones. A higher SMI means more rod-like trabeculae. Rod-like trabeculae may have more coverage for other trabeculae (though less coverage for marrow).

Finally, the old DPF 40 NICD mice showed an increased bone volume fraction and trabecular thickness. (FIG. 17B; DPF 40) At 40 DPF a murine tibial fracture is remodeling and the actual fracture healing is over. Since the callus has an increased bone volume fraction and trabecular thickness, it can be concluded that NICD makes the callus stronger and able to withstand more force, which is especially vital in the geriatric bone.

A goal of this study was to see if geriatric fractures could benefit from NICD injection. This appears to be true, since NICD effectively stimulates early calcification through the stimulation of hypertrophic chondrocytes, meaning a more robust callus early on which means earlier weight bearing. It also brings forth a callus with a higher amount of calcium and bone in the late stages of fracture healing, meaning the bone is less brittle.

These results clearly show a positive effect of Notch activation on both callus size and bone formation, particularly in the geriatric mice.

TABLE 1
Histology results. NICD vs GFP in young and old
mice, seen on 10, 20 and 40 DPF. Means are shown

| | | | Total callus | Cartilage | Cartilage |
|---|---|---|---|---|---|
| DPF | Age | V/P (n = . . . ) | area | area | percentage |
| 10 DPF | Young | NICD n = 2/3* | 58.48 | 13.39 | 26.66% |
| | | GFP n = 4 | 55.51 | 8.10 | 19.86% |
| | Old | NICD n = 3 | 65.24 | 7.07 | 10.96% |
| | | GFP n = 3 | 34.98 | 5.05 | 15.02% |
| 20 DPF | Young | NICD n = 4 | 57.14 | 1.59 | 2.58% |
| | | GFP n = 4 | 66.34 | 4.30 | 5.81% |
| | Old | NICD n = 4 | 77.81 | 6.84 | 8.43% |
| | | GFP n = 4 | 58.57 | 2.57 | 4.78% |
| 40 DPF | Young | NICD n = 5 | 72.29 | NO CARTILAGE | NO CARTILAGE |
| | | GFP n = 5 | 52.27 | NO CARTILAGE | NO CARTILAGE |
| | Old | NICD n = 4 | 60.69 | NO CARTILAGE | NO CARTILAGE |
| | | GFP n = 4 | 58.32 | NO CARTILAGE | NO CARTILAGE |

*3 samples for callus area. 2 samples for cartilage area and - percentage due to no cartilage in callus (cut not deep enough, stain worked, since cartilaginous part of joint was stained)
N = number of samples.
NICD = Notch Intracellular Domain.
GFP = Green Fluorescent protein

TABLE 2
MicroCT results. NICD vs GFP in young and old mice, seen on DPF 10, 20 and 40. Means are shown.

| | | | | | | Tissue | | | | | Structure |
|---|---|---|---|---|---|---|---|---|---|---|---|
| | | NICD/ | Total callus | Total bone | Bone volume | mineral | Trabecular | Trabecular | Trabecular | Conn. | Model |
| DPF | Age | GFP | volume | volume | fraction | density | number | thickness | separation | density | Index |
| 10 | Young | NICD | XXXXX* | 0.956 | XXXXX* | 304.739 | 0.849 | 0.095 | 1.475 | 0.955 | 2.303 |
| | | (N = 7) | | | | | | | | | |
| | | GFP | XXXXX* | 0.915 | XXXXX* | 307.041 | 0.742 | 0.095 | 1.603 | 0.668 | 2.219 |
| | | (N = 7) | | | | | | | | | |
| | Old | NICD | XXXXX* | 0.825 | XXXXX* | 415.684 | 0.607 | 0.095 | 1.902 | 0.397 | 2.372 |
| | | (N = ?) | | | | | | | | | |
| | | GFP | XXXXX* | 0.540 | XXXXX* | 306.020 | 0.619 | 0.063 | 1.787 | 0.573 | 2.094 |
| | | (N = ?) | | | | | | | | | |
| 20 | Young | NICD | 37.243^{†} | 15.367 | 0.402 | 359.279 | 4.580 | 0.150 | 0.247 | 98.608 | 0.703 |
| | | (N = 7) | | | | | | | | | |
| | | GFP | 30.376 | 12.335 | 0.416 | 357.358 | 4.638 | 0.141 | 0.256 | 103.600 | 0.596 |
| | | (N = 7) | | | | | | | | | |
| | Old | NICD | 26.000^{†} | 8.722 | 0.334 | 330.433 | 4.092 | 0.113 | 0.305 | 105.339 | 1.327 |
| | | (N = 6)^{‡} | | | | | | | | | |
| | | GFP | 21.112 | 7.354 | 0.361 | 334.439 | 4.620 | 0.111 | 0.253 | 115.023 | 1.265 |
| | | (N = 6)^{‡} | | | | | | | | | |
| 40 | Young | NICD | 27.031 | 9.311 | 0.348 | 680.859^{†} | 3.130 | 0.153 | 0.343 | 39.828 | 1.495 |
| | | (N = 7) | | | | | | | | | |
| | | GFP | 23.639 | 8.413 | 0.373 | 635.887 | 3.372 | 0.145 | 0.323 | 41.318 | 1.020 |
| | | (N = 7) | | | | | | | | | |
| | Old | NICD | 17.760 | 6.422 | 0.361^{†} | 520.913 | 3.784^{†} | 0.124 | 0.293 | 57.079 | 1.014 |
| | | (N = 7) | | | | | | | | | |
| | | GFP | 18.437 | 4.727 | 0.259 | 528.789 | 3.555 | 0.096 | 0.319 | 50.141 | 1.723 |
| | | (N = 7) | | | | | | | | | |

*No macroscopic calcification on DPF 10, therefore, a square as outline and measured calcification within.
^{†}Significant (p = <0.05) between NICD and GFP (the ^{†} is only shown at NICD)
^{‡}There was an error in one of the scans, by the time it was observed it had already been decalcified.
N = number of samples.
NICD = Notch Intracellular Domain.
GFP = Green Fluorescent protein

Referenced Sequences

| SEQ | | |
|---|---|---|
| ID | | |
| NO | Name | Sequeuce |
| 6 | Human | ATGtcccgcaagcgccggcggcagcatggccagctctggttccctgagggcttcaaagtgtctg |
| | NICD1 | aggccagcaagaagaagcggcgggagcccctcggcgaggactccgtgggcctcaagcccctga |
| | cDNA seq. | agaacgcttcagacggtgccctcatggacgacaaccagaatgagtggggggacgaggacctgga |
| | (includes a | gaccaagaagttccggttcgaggagcccgtggttctgcctgacctggacgaccagacagaccacc |
| | starting | ggcagtggactcagcagcacctggatgccgctgacctgcgcatgtctgccatggcccccacaccg |
| | ATG) | ccccagggtgaggttgacgccgactgcatggacgtcaatgtccgcgggcctgatggcttcaccccg |
| | | ctcatgatcgcctcctgcagcgggggcggcctggagacgggcaacagcgaggaagaggaggac |
| | | gcgccggccgtcatctccgacttcatctaccagggcgccagcctgcacaaccagacagaccgcac |
| | | gggcgagaccgccttgcacctggccgcccgctactcacgctctgatgccgccaagcgcctgctgg |
| | | aggccagcgcagatgccaacatccaggacaacatgggccgcaccccgctgcatgcggctgtgtct |
| | | gccgacgcacaaggtgtcttccagatcctgatccggaaccgagccacagacctggatgcccgcat |
| | | gcatgatggcacgacgccactgatcctggctgcccgcctggccgtggagggcatgctggaggacc |
| | | tcatcaactcacacgccgacgtcaacgccgtagatgacctgggcaagtccgccctgcactgggcc |
| | | gccgccgtgaacaatgtggatgccgcagttgtgctcctgaagaacggggctaacaaagatatgcag |
| | | aacaacagggaggagacacccctgtttctggccgcccgggagggcagctacgagaccgccaag |
| | | gtgctgctggaccactttgccaaccgggacatcacggatcatatggaccgcctgccgcgcgacatc |
| | | gcacaggagcgcatgcatcacgacatcgtgaggctgctggacgagtacaacctggtgcgcagccc |
| | | gcagctgcacggagccccgctggggggcacgcccaccctgtcgcccccgctctgctcgcccaac |
| | | ggctacctgggcagcctcaagcccggcgtgcagggcaagaaggtccgcaagcccagcagcaaa |
| | | ggcctggcctgtggaagcaaggaggccaaggacctcaaggcacggaggaagaagtcccaggac |
| | | ggcaagggctgcctgctggacagctccggcatgctctcgcccgtggactccctggagtcaccccat |
| | | ggctacctgtcagacgtggcctcgccgccactgctgccctccccgttccagcagtctccgtccgtgc |
| | | ccctcaaccacctgcctgggatgcccgacacccacctgggcatcgggcacctgaacgtggcggcc |
| | | aagcccgagatggcggcgctgggtgggggcggccggctggcctttgagactggcccacctcgtct |
| | | ctcccacctgcctgtggcctctggcaccagcaccgtcctgggctccagcagcggaggggccctga |
| | | atttcactgtgggcgggtccaccagtttgaatggtcaatgcgagtggctgtcccggctgcagagcgg |
| | | catggtgccgaaccaatacaaccctctgcgggggagtgtggcaccaggccccctgagcacacag |
| | | gccccctccctgcagcatggcatggtaggcccgctgcacagtagccttgctgccagcgccctgtcc |
| | | cagatgatgagctaccagggcctgcccagcacccggctggccacccagcctcacctggtgcagac |
| | | ccagcaggtgcagccacaaaacttacagatgcagcagcagaacctgcagccagcaaacatccag |
| | | cagcagcaaagcctgcagccgccaccaccaccaccacagccgcaccttggcgtgagctcagcag |
| | | ccagcggccacctgggccggagcttcctgagtggagagccgagccaggcagacgtgcagccact |
| | | gggccccagcagcctggcggtgcacactattctgccccaggagagccccgccctgcccacgtcg |
| | | ctgccatcctcgctggtcccacccgtgaccgcagcccagttcctgacgcccccctcgcagcacagc |
| | | tactcctcgcctgtggacaacacccccagccaccagctacaggtgcctgagcaccccttcctcaccc |
| | | cgtcccctgagtcccctgaccagtggtccagctcgtccccgcattccaacgtctccgactggtccga |
| | | gggcgtctccagccctcccaccagcatgcagtcccagatcgcccgcattccggaggccttcaagta |
| | | a |
| 7 | Human | Msrkrrrqhgqlwfpegfkvseaskkkrreplgedsvglkplknasdgalmddnqnewgde |
| | NICD1 | dletkkfrfeepvvlpdlddqtdhrqwtqqhldaadlrmsamaptppqgevdadcmdvnvrg |
| | protein seq. | pdgftplmiascsgggletgnseeeedapavisdfiyqgaslhnqtdrtgetalhlaarysrsdaak |
| | (includes a | rlleasadaniqdnmgrtplhaavsadaqgvfqilirnratdldarmhdgttplilaarlavegmle |
| | starting met.) | dlinshadvnavddlgksalhwaaavnnvdaavvllkngankdmqnnreetplflaaregsye |
| | | takvlldhfanrditdhmdrlprdiaqermhhdivrlldeynlvrspqlhgaplggtptlspplcsp |
| | | ngylgslkpgvqgkkvrkpsskglacgskeakdlkarrkksqdgkgclldssgmlspvdslesp |
| | | hgylsdvasppllpspfqqspsvplnhlpgmpdthlgighlnvaakpemaalggggrlafetgp |
| | | prlshlpvasgtstvlgsssggalnftvggstslngqcewlsrlqsgmvpnqynplrgsvapgpls |
| | | tqapslqhgmvgplhsslaasalsqmmsyqglpstrlatqphlvqtqqvqpqnlqmqqqn1qp |
| | | aniqqqqslqppppppqphlgvssaasghlgrsflsgepsqadvqplgpsslavhtilpqespal |
| | | ptslpsslvppvtaaqfltppsqhsysspvdntpshqlqvpehpfltpspespdqwssssphsnv |
| | | sdwsegvsspptsmqsqiaripeafk |
| 8 | Human | ATGgcaaaacgaaagcgtaagcatggctctctctggctgcctgaaggtttcactcttcgccgagat |
| | NICD2 | gcaagcaatcacaagcgtcgtgagccagtgggacaggatgctgtggggctgaaaaatctctcagtg |
| | cDNA seq. | caagtctcagaagctaacctaattggtactggaacaagtgaacactgggtcgatgatgaagggccc |
| | (includes a | cagccaaagaaagtaaaggctgaagatgaggccttactctcagaagaagatgaccccattgatcga |
| | starting | cggccatggacacagcagcaccttgaagctgcagacatccgtaggacaccatcgctggctctcacc |
| | ATG) | cctcctcaggcagagcaggaggtggatgtgttagatgtgaatgtccgtggcccagatggctgcacc |
| | | ccattgatgttggcttctctccgaggaggcagctcagatttgagtgatgaagatgaagatgcagagg |
| | | actcttctgctaacatcatcacagacttggtctaccagggtgccagcctccaggcccagacagaccg |
| | | gactggtgagatggccctgcaccttgcagcccgctactcacgggctgatgctgccaagcgtctcctg |
| | | gatgcaggtgcagatgccaatgcccaggacaacatgggccgctgtccactccatgctgcagtggc |
| | | agctgatgcccaaggtgtcttccagattctgattcgcaaccgagtaactgatctagatgccaggatga |
| | | atgatggtactacacccctgatcctggctgcccgcctggctgtggagggaatggtggcagaactgat |
| | | caactgccaagcggatgtgaatgcagtggatgaccatggaaaatctgctcttcactgggcagctgct |
| | | gtcaataatgtggaggcaactcttttgttgttgaaaaatggggccaaccgagacatgcaggacaaca |
| | | aggaagagacacctctgtttcttgctgcccgggaggggagctatgaagcagccaagatcctgttag |
| | | accattttgccaatcgagacatcacagaccatatggatcgtcttccccgggatgtggctcgggatcgc |
| | | atgcaccatgacattgtgcgccttctggatgaatacaatgtgaccccaagccctccaggcaccgtgtt |
| | | gacttctgctctctcacctgtcatctgtgggcccaacagatctttcctcagcctgaagcacaccccaat |
| | | gggcaagaagtctagacggcccagtgccaagagtaccatgcctactagcctccctaaccttgccaa |
| | | ggaggcaaaggatgccaagggtagtaggaggaagaagtctctgagtgagaaggtccaactgtctg |
| | | agagttcagtaactttatcccctgttgattccctagaatctcctcacacgtatgtttccgacaccacatcc |
| | | tctccaatgattacatcccctgggatcttacaggcctcacccaaccctatgttggccactgccgcccct |
| | | cctgccccagtccatgcccagcatgcactatctttttctaaccttcatgaaatgcagcctttggcacatg |
| | | gggccagcactgtgcttccctcagtgagccagttgctatcccaccaccacattgtgtctccaggcagt |
| | | ggcagtgctggaagcttgagtaggctccatccagtcccagtcccagcagattggatgaaccgcatg |
| | | gaggtgaatgagacccagtacaatgagatgtttggtatggtcctggctccagctgagggcacccatc |
| | | ctggcatagctccccagagcaggccacctgaagggaagcacataaccacccctcgggagcccttg |
| | | ccccccattgtgactttccagctcatccctaaaggcagtattgcccaaccagcgggggctccccagc |
| | | ctcagtccacctgccctccagctgttgcgggccccctgcccaccatgtaccagattccagaaatggc |
| | | ccgtttgcccagtgtggctttccccactgccatgatgccccagcaggacgggcaggtagctcagac |
| | | cattctcccagcctatcatcctttcccagcctctgtgggcaagtaccccacacccccttcacagcaca |
| | | gttatgcttcctcaaatgctgctgagcgaacacccagtcacagtggtcacctccagggtgagcatcc |
| | | ctacctgacaccatccccagagtctcctgaccagtggtcaagttcatcaccccactctgcttctgactg |
| | | gtcagatgtgaccaccagccctacccctgggggtgctggaggaggtcagcggggacctgggaca |
| | | cacatgtctgagccaccacacaacaacatgcaggtttatgcgtga |
| 9 | Human | Makrkrkhgslwlpegftlrrdasnhkrrepvgqdavglknlsvqvseanligtgtsehwvdde |
| | NICD2 | gpqpkkvkaedeallseeddpidrrpwtqqhleaadirrtpslaltppqaeqevdvldvnvrgpd |
| | protein seq. | gctplmlaslrggssdlsdededaedssaniitdlvyqgaslqaqtdrtgemalhlaarysradaak |
| | (includes a | rlldagadanaqdnmgrcplhaavaadaqgvfqilirnrvtdldarmndgttplilaarlavegm |
| | starting met.) | vaelincqadvnavddhgksalhwaaavnnveatllllknganrdmqdnkeetplflaaregsy |
| | | eaakilldhfanrchtdhmdrlprdvardrmhhdivrlldeynvtpsppgtvltsalspvicgpnrs |
| | | flslkhtpmgkksrrpsakstmptslpnlakeakdakgsrrkkslsekvqlsessvtlspvdslesp |
| | | htyvsdttsspmitspgilqaspnpmlataappapvhaqhalsfsnlhemqplahgastvlpsvs |
| | | qllshhhivspgsgsagslsrlhpvpvpadwmnrmevnetqynemfgmvlapaegthpgia |
| | | pqsrppegkhittpreplppivtfqlipkgsiaqpagapqpqstcppavagplptmyqipemarl |
| | | psvafptammpqqdgqvaqtilpayhpfpasvgkyptppsqhsyassnaaertpshsghlqge |
| | | hpyltpspespdqwssssphsasdwsdvttsptpggagggqrgpgthmsepphnnmqvya |
| 10 | Mouse | ATGtcccgcaagcgccggcggcagcatggccagctctggttccctgagggtttcaaagtgtcag |
| | NICD1 | aggccagcaagaagaagcggagagagcccctcggcgaggactcagtcggcctcaagcccctga |
| | cDNA seq. | agaatgcctcagatggtgctctgatggacgacaatcagaacgagtggggagacgaagacctggag |
| | (includes a | accaagaagttccggtttgaggagccagtagttctccctgacctgagtgatcagactgaccacaggc |
| | starting | agtggacccagcagcacctggacgctgctgacctgcgcatgtctgccatggccccaacaccgcct |
| | ATG) | cagggggaggtggatgctgactgcatggatgtcaatgttcgaggaccagatggcttcacacccctc |
| | | atgattgcctcctgcagtggagggggccttgagacaggcaacagtgaagaagaagaagatgcacc |
| | | tgctgtcatctctgacttcatctaccagggcgccagcttgcacaaccagacagaccgcaccgggga |
| | | gaccgccttgcacttggctgcccgatactctcgttcagatgctgcaaagcgcttgctggaggccagt |
| | | gcagatgccaacatccaggacaacatgggccgtactccgttacatgcagcagtttctgcagatgctc |
| | | agggtgtcttccagatcctgctccggaacagggccacagatctggatgcccgaatgcatgatggca |
| | | caactccactgatcctggctgcgcgcctggccgtggagggcatgctggaggacctcatcaactcac |
| | | atgctgacgtcaatgccgtggatgacctaggcaagtcggctttgcattgggcggccgcggtgaaca |
| | | atgtggatgctgctgttgtgctcctgaagaacggagccaacaaggacatgcagaacaacaaggag |
| | | gagactcccctgttcctggccgcccgtgagggcagctatgagactgccaaagtgttgctggaccact |
| | | ttgccaaccgggacatcacggatcacatggaccgattgccgcgggacatcgcacaggagcgtatg |
| | | caccacgatatcgtgcggcttttggatgagtacaacctggtgcgcagcccacagctgcatggcactg |
| | | ccctgggtggcacacccactctgtctcccacactctgctcgcccaatggctacctgggcaatctcaa |
| | | gtctgccacacagggcaagaaggcccgcaagcccagcaccaaagggctggcttgtggtagcaag |
| | | gaagctaaggacctcaaggcacggaggaagaagtcccaggatggcaagggctgcctgttggaca |
| | | gctcgagcatgctgtcgcctgtggactccctcgagtcaccccatggctacttgtcagatgtggcctcg |
| | | ccacccctcctcccctccccattccagcagtctccatccatgcctctcagccacctgcctggtatgcct |
| | | gacactcacctgggcatcagccacttgaatgtggcagccaagcctgagatggcagcactggctgg |
| | | aggtagccggttggcctttgagccacccccgccacgcctctcccacctgcctgtagcctccagtgcc |
| | | agcacagtgctgagtaccaatggcacgggggctatgaatttcaccgtgggtgcaccggcaagcttg |
| | | aatggccagtgtgagtggcttccccggctccagaatggcatggtgcccagccagtacaacccacta |
| | | cggccgggtgtgacgccgggcacactgagcacacaggcagctggcctccagcatagcatgatgg |
| | | ggccactacacagcagcctctccaccaataccttgtccccgattatttaccagggcctgcccaacac |
| | | acggctggcaacacagcctcacctggtgcagacccagcaggtgcagccacagaacttacagctcc |
| | | agcctcagaacctgcagccaccatcacagccacacctcagtgtgagctcggcagccaatgggcac |
| | | ctgggccggagcttcttgagtggggagcccagtcaggcagatgtacaaccgctgggccccagcag |
| | | tctgcctgtgcacaccattctgccccaggaaagccaggccctgcccacatcactgccatcctccatg |
| | | gtcccacccatgaccactacccagttcctgacccctccttcccagcacagttactcctcctcccctgtg |
| | | gacaacacccccagccaccagctgcaggtgccagagcaccccttcctcaccccatcccctgagtc |
| | | ccctgaccagtggtccagctcctccccgcattccaacatctctgattggtccgagggcatctccagcc |
| | | cgcccaccaccatgccgtcccagatcacccacattccagaggcatttaaataa |
| 11 | Mouse | Msrkrrrqhgqlwfpegfkvseaskkkrreplgedsvglkplknasdgalmddnqnewgde |
| | NICD1 | dletkkfrfeepvvlpdlsdqtdhrqwtqqhldaadlrmsamaptppqgevdadcmdvnvrg |
| | protein seq. | pdgftplmiascsgggletgnseeeedapavisdfiyqgaslhnqtdrtgetalhlaarysrsdaak |
| | (includes a | rlleasadaniqdnmgrtplhaavsadaqgvfqillrnratdldarmhdgttplilaarlavegmle |
| | starting met.) | dlinshadvnavddlgksalhwaaavnnvdaavvllkngankdmqnnkeetplflaaregsye |
| | | takvlldhfanrditdhmdrlprdiaqermhhdivlldeynlvrspqlhgtalggtptlsptlcspn |
| | | gylgnlksatqgkkarkpstkglacgskeakdlkarrkksqdgkgclldsssmlspvdslesphg |
| | | ylsdvasppllpspfqqspsmplshlpgmpdthlgishlnvaakpemaalaggsrlafeppppr |
| | | lshlpvassastvlstngtgamnftvgapashigqcewlprlqngmvpsqynplrpgvtpgtlst |
| | | qaaglqhsmmgplhsslstntlspiiyqglpntrlatqphlvqtqqvqpqnlqlqpqnlqppsqp |
| | | hlsvssaanghlgrsflsgepsqadvqplgpsslpvhtilpqesqalptslpssmvppmtttqfltp |
| | | psqhsyssspvdntpshqlqvpehpfltpspespdqwssssphsnisdwsegissppttmpsqi |
| | | thipeafk |
| 12 | Mouse | ATGgccaagcggaagcgcaagcatggcttcctctggctgccggaagggttcaccctccgccga |
| | NICD2 | gactctagcaatcacaagcgccgtgaacctgtgggacaggatgccgtggggctgaaaaatctctcc |
| | cDNA seq. | gtgcaagtgtcagaggctaacctgattggttctgggacaagtgaacattgggttgatgatgaaggac |
| | (includes a | cccagccaaagaaagccaaggctgaggatgaggctttgctgtcggaagatgaccccatcgatcga |
| | starting | cggccctggacacagcagcaccttgaagctgcagacatccgccacactccatccctggcactcact |
| | ATG) | cctcctcaggcagaacaggaggtggacgtgctggacgtgaatgtccgaggcccagatgggtgtac |
| | | tccactgatgctggcttctctccgaggaggcagctcagacctgagtgatgaagacgaagatgctga |
| | | ggactcttctgccaacatcatcacagacttggtctaccaaggtgccagccttcaagcacagacagac |
| | | cgcactggcgagatggccctgcaccttgcagcccgctattcgagagctgatgctgccaaacgcctc |
| | | ctggatgctggtgcggatgcaaatgcccaggacaacatgggccgatgtcctcttcacgctgcggtg |
| | | gcagcagacgcccaaggtgtctttcagattctgatccgcaaccgtgtaaccgatctggatgccagaa |
| | | tgaacgatggtactacccccctgatcctggctgcccgcctggctgtggaaggaatggtggcagagtt |
| | | gatcaattgccaagcagatgtcaatgcagtggatgaccatggaaaatctgccctccactgggcagct |
| | | gctgtcaataatgtggaggcgactcttctgctgttgaagaatggggccaacagagatatgcaggaca |
| | | ataaggaagagacacctttgtttcttgctgcccgagagggaagttatgaagcggccaaaatcctgtta |
| | | gaccattttgccaaccgggacatcactgaccacatggaccgccttccccgggatgtggctcgggac |
| | | cgcatgcaccatgacatcgttcgcctcctggacgagtataacgtgactcccagccctccgggaacg |
| | | gtcttgacttctgcgctctcacctgtcctctgtgggcccaacaggtctttcctcagtctgaagcacaccc |
| | | caatgggtaagaaggctaggcggcccaacaccaagagcaccatgcccacgagcctgcctaacctt |
| | | gccaaggaggccaaggatgccaagggcagcaggaggaagaagtgtctgaacgagaaggtccag |
| | | ctgtccgagagctcagtgactctatcccccgtcgattcgctcgagtctcctcacacgtatgtctccgat |
| | | gccacatcctctcccatgatcacatcccctggaatcttacaggcctcgcccacccccctgctggctgc |
| | | tgccgccccggctgccccagtgcacacacagcatgcgctgtctttctctaaccttcatgacatgcagc |
| | | ctttggctcctggagccagcaccgtgctcccctcggtcagccagctgctatcccaccaccacatcgc |
| | | gcccccaggtagtagcagtgcaggaagcttgggcaggttacatccagttcctgtcccagcagactg |
| | | gatgaaccgtgtggagatgaacgagacccagtacagtgaaatgtttggcatggtcctggctcctgca |
| | | gagggagcccaccctggcatagcagctccccagagcagacctccggaagggaagcacatgtcca |
| | | cccagcgggagcccttgcctcccatcgtgactttccagcttatcccaaaaggcagcattgcccaggc |
| | | agccggagctccccagacgcagtccagttgccctccagctgttgcaggccccttgccctctatgtac |
| | | cagatcccagagatgccccgtttgcccagtgtggctttcccacctaccatgatgccccagcaggagg |
| | | ggcaggtagctcagaccattgtgccaacctatcatcctttcccagcctctgtgggcaagtaccccaca |
| | | cccccttcccaacacagttacgcctcctcaaatgctgctgagcgaacccccagtcatggtggtcacct |
| | | ccagggcgagcacccatacctgacaccatccccagagtctcctgaccaatggtcaagctcttcacc |
| | | acactctgcatctgactggtcagatgtgaccaccagcccaactcctggaggtggtggaggcggtca |
| | | gcggggacccggaacacacatgtccgagccaccacacagcaacatgcaggtgtatgcatga |
| 13 | Mouse | Makrkrkhgflwlpegftlrrdssnhkrrepvgqdavglknlsvqvseanligsgtsehwvdde |
| | NICD2 | gpqpkkakaedeallseddpidrrpwtqqhleaadirhtpslaltppqaeqevdvldvnvrgpd |
| | protein seq. | gctplmlaslrggssdlsdededaedssaniitdlvyqgaslqaqtdrtgemalhlaarysradaak |
| | includes a | rlldagadanaqdnmgrcplhaavaadaqgvfqilirnrvtdldarmndgttplilaarlavegm |
| | starting met.) | vaelnicqadvnavddhgksalhwaaavnnveatllllknganrdmqdnkeetplflaaregsy |
| | | eaakilldhfanrditdhmdrlprdvardrmhhdivrlldeynvtpsppgtvltsalspvlcgpnrs |
| | | flslkhtpmgkkarrpntkstmptslpnlakeakdakgsrrkkclnekvqlsessvtlspvdsles |
| | | phtyvsdatsspmitspgilqasptpllaaaapaapvhtqhalsfsnlhdmqplapgastvlpsvs |
| | | qllshhhiappgsssagslgrlhpvpvpadwmnrvemnetqysemfgmvlapaegahpgia |
| | | apqsrppegkhmstqreplppivtfqlipkgsiaqaagapqtqsscppavagplpsmyqipem |
| | | prlpsvafpptmmpqqegqvaqlivptyhpfpasvgkyptppsqhsyassnaaertpshgghl |
| | | qgehpyltpspespdqwssssphsasdwsdvttsptpggggggqrgpgthmsepphsnmqv |
| | | ya |

### Example 10

### Notch1 and Notch2 Receptors Show Opposite Patterns of Expression and Differing Effects on Osteoblastogenesis in Murine and Human Mesenchymal Stem Cells

The expression of Notch1 and Notch2 in multiple donor-lines of primary hMSC and mMSC was investigated using quantitative PCR. Notch2 expression level was 40-fold greater than Notch1 in undifferentiated hMSC, while in mMSC the expression level of Notch2 was much lower than that of Notch1 (0.15-fold decrease) (data not shown). This elevated Notch1 relative to Notch2 is similar in mouse cortical bone (0.2-fold Notch2 relative to Notch1); however, when bone is injured, during the course of healing, the levels of Notch2 increase relative to Notch1 temporally. Thus, the NICD2/NICD1 ratio is increased to 0.4 relative to Notch1 at day 5 post-fracture, 2 at day 10 post-fracture, and 4 at day 20 post-fracture (data not shown).

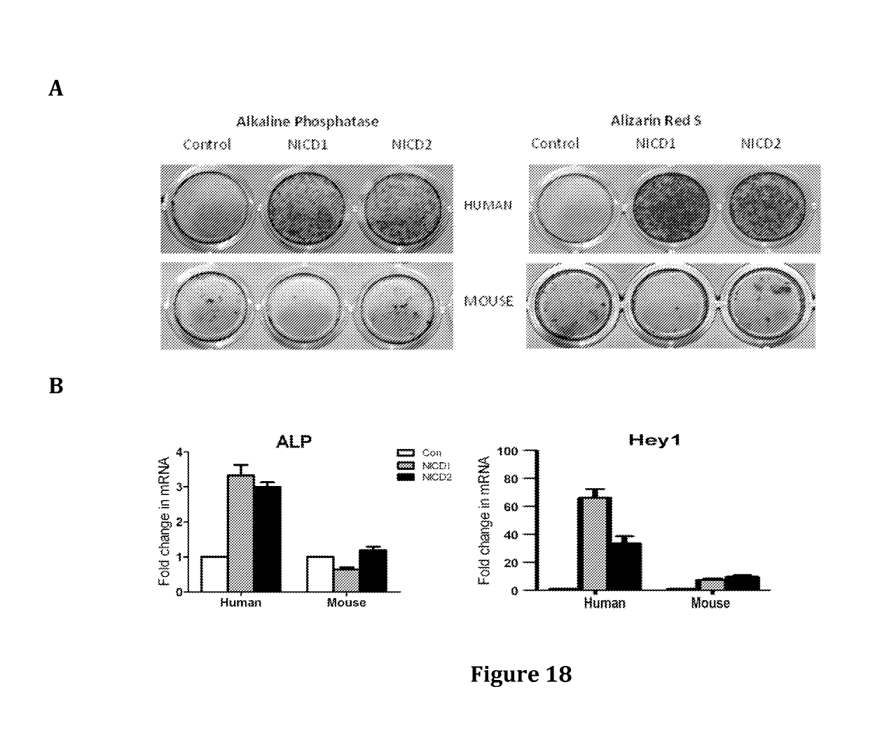

The impact of overexpressing the active intracellular domains of Notch1 (NICD1) and Notch2 (NICD2) in both primary mMSC and hMSC was evaluated using retrovirus. Human or mouse MSC were transfected with NICD1, NICD2, or control viral supernatants, and then cultured for 48 hrs on plates with osteogenic medium including BGP and Ascorbic. In FIG. 18A, the cells were stained for alkaline phosphatase activity (ALP) at day 5 (left panel, purple) and stained with Alizarin Red S at day 10 for calcium mineral (right panel, red). Both NICD1 and NICD2 increase human MSC osteoblast differentiation, while NICD1 inhibits murine MSC osteoblast differentiation. Additionally, NICD1 and NICD2 overexpression in either mMSC or hMSC significantly upregulated the canonical Notch target gene Hey1 (FIG. 18B). While both NICD1 and NICD2 increase Hey1 expression for mouse and human, indicating functional Notch signaling, NICD1 and NICD2 only positively influence alkaline phosphatase (ALP) gene expression of human cells. Thus, both NICD1 and NICD2 are sufficient to induce osteoblast gene expression and mineralization in hMSC. In contrast, overexpression of NICD1 inhibited osteoblast gene expression and mineralization in mMSC, while NICD2 overexpression in mMSC showed no negative effects on osteoblastogenesis.

These results suggest that Notch1 and Notch2 have opposite patterns of expression in primary mouse and human MSC. Particularly, Notch1 is dominant in mMSC while Notch2 is dominant in hMSC. During fracture healing in mice, Notch1 and Notch2 expression shift during the osteogenic phase of bone regeneration. Intriguingly, when NICD1 is overexpressed in mMSC, it has the same deleterious effect on osteoblastogenesis as observed when mMSC were plated on Jagged-1. This suggests that the Notch1 expression dominance in mMSC may limit osteoblastogenesis when cells are exposed to Jagged-1.

What is claimed:

1. An implantable composition comprising: an osteoinductive Notch ligand bound to at least one biocompatible substrate.

2. The composition of claim 1, wherein the biocompatible substrate comprises a calcium-based ceramic, phosphate-based ceramic, allograft, autograft, polymer, bioglass, collagen-based material, proteoglycan-based material, glycosaminoglycan-based material, or polysaccharide.

3. The composition of claim 2, wherein the substrate comprises a poly(β-amino ester).

4. The composition of claim 3, wherein the substrate comprises a diacrylate and an amine.

5. The composition of claim 4, wherein the substrate comprises diethylene glycol diacrylate and isobutylamine.

6. The composition of any one of the previous claim, wherein the substrate is biodegradable.

7. The composition of any one of claims 1 to 5, wherein the substrate is non-biodegradable.

8. The composition of any one of the previous claims, wherein the substrate comprises a solid support, aqueous solution, gel, hydrogel, bead, microbead, nanoparticle, or any combination thereof.

9. The composition of any one of the previous claims, wherein the Notch ligand activates the Notch signaling pathway.

10. The composition of any one of the previous claims, wherein the Notch ligand is delivered to a site in need of bone tissue formation.

11. The composition of claim 10, wherein the bone tissue formation occurs through endochondral bone regeneration, intramembranous bone regeneration, or any combination thereof.

12. The composition of any one of the previous claims, wherein the Notch ligand is derived from any mammalian species.

13. The composition of any one of the previous claims, wherein the Notch ligand comprises full length Jagged-1 protein.

14. The composition of any one of claims 1 to 12, wherein the Notch ligand comprises a portion of full length Jagged-1 protein.

15. The composition of any one of the previous claims, wherein the Jagged-1 protein comprises a native form, a variant, a recombinant, an analog, a derivative, a chimeric, or any combination thereof.

16. The composition of any one of the previous claims, wherein the Notch ligand comprises a fusion protein.

17. The composition of any one of the previous claims, wherein the Notch ligand is sufficiently present to induce expression of Notch target genes.

18. The composition of claim 17, wherein the target genes comprise Hey1, Hes1, or any combination thereof.

19. The composition of any one of the previous claims, wherein the Notch ligand is sufficiently present to increase gene expression of osteogenic differentiation markers.

20. The composition of claim 19, wherein the osteogenic differentiation marker comprise bone sialoprotein, alkaline phosphatase, or any combination thereof.

21. The composition of any one of the previous claims, wherein the Notch ligand is sufficiently present to increase the activity of osteogenic enzymes.

22. The composition of claim 21, wherein the enzyme comprises alkaline phosphatase.

23. The composition of any one of the previous claims, wherein the Notch ligand is sufficiently present to result in the production of a hydroxyapatite mineralized matrix.

24. The composition of any one of the previous claims, wherein the ligand is directly bound to the substrate.

25. The composition of claim 24, wherein the ligand is covalently bound to said substrate.

26. The composition of claim 24, wherein the ligand is bound to said substrate through adsorption.

27. The composition of any of the previous claims, wherein the ligand is indirectly bound to said substrate.

28. The composition of claim 27, wherein the ligand is indirectly bound through a linker.

29. The composition of claim 28, wherein the linker is covalently bound to the scaffold.

30. The composition of claim 28, wherein the linker is bound to the scaffold through adsorption.

31. The composition of claim 27, wherein the ligand is indirectly bound through an antibody.

32. The composition of claim 31, wherein the antibody is an anti-Fc antibody.

33. The composition of claim 31, wherein the antibody is an antibody specific for the Notch ligand.

34. The composition of claim 33, wherein the antibody is an anti-Jagged-1 antibody.

35. The composition of claim 33, wherein the antibody recognizes a portion of the Notch ligand that does not prevent receptor binding, activation, or both.

36. The composition of any one of the previous claims, further comprising cells.

37. The composition of claim 36, wherein the cells comprise osteogenic cells.

38. The composition of claim 37, wherein the cells comprise mesenchymal stem cells, embryonic stem cells, induced pluripotent stem cells, or any combination thereof.

39. The composition of claim 36, wherein the cells are derived from embryonic stem cells, induced pluripotent stem cells, or any combination thereof.

40. The composition of any of claims 36 to 39, wherein the cells are harvested from bone marrow, adipose tissue, periosteal tissue, muscle tissue, umbilical cord, blood, or any combination thereof.

41. The composition of any one of claims 36 to 39, wherein the cells are attached to the biomaterial substrate and interact with the Notch ligand.

42. The composition of claim 41, wherein the Notch ligand promotes differentiation of the cells into osteoblasts.

43. The composition of any one of the previous claims, wherein the composition is capable of implantation into a patient in need of bone tissue formation.

44. The composition of claim 43, wherein cells bound to the Notch ligand bound biomaterial substrate are expanded ex vivo prior to implantation.

45. The composition of claim 43, wherein the composition is implanted without ex vivo expansion of the cells.

46. A method of treating a patient in need of bone tissue formation comprising: administering a composition of any one of the previous claims to a patient in need of treatment.

47. The method of claim 46, comprising administering the composition in a bone, proximate to a bone, or any combination thereof.

48. The method of claim 46, wherein the composition is used to treat fractures, breaks, spinal fusion, damage due to tissue removal, facial reconstruction, dental reconstruction, distraction osteogenesis, joint revision, osteolysis, osteoporosis, genetic disease, or any combination thereof.

49. The method of any one of claims 46 to 48, wherein the administered composition interacts with patient cells following administration of the composition.

50. The method of claim 49, wherein the patient's cells bind to the administered composition.

51. The method of any one of claims 46 to 50, wherein the administered composition induces cells to differentiate into osteoblasts following binding to the composition.

52. The method of claim 51, wherein said osteoblasts produce bone tissue.

53. The method of claim 52, wherein the bone tissue is incorporated into or proximate to the bone in need of treatment.

54. The method of any one of claims 46 to 53, wherein the substrate degrades following incorporation of bone tissue into a site of treatment.

55. The method of any one of claims 46 to 53, wherein the substrate does not degrade following incorporation of bone tissue into a site of treatment.

56. A method of treating a patient in need of bone tissue formation comprising: administering to said patient a composition comprising a nucleic acid molecule encoding a notch intracellular domain (NICD).

57. The method of claim 56, wherein administration of said NICD increases callus volume, bone volume fraction, trabecular thickness, or any combination thereof.

57. A kit comprising: a pharmaceutically acceptable package containing the composition of any one of claims 1 to 45.
